SEQUENCE LISTING (20 sequences)

SEQ ID NO: 1 - protein, 276 aa, Staphylococcus aureus
MGSSHHHHHHSSGLVPRGSHMFKKLINKKNTINNYNEELDSSNIPEHIAIIMDGNGRWAKKRKMPRIKGHYEGMQTIKKITRVASDIGVKYLTLYAFSTENWSRPESEVNYIMNLPVNFLKTFLPELIEKNVKVETIGFTDKLPKSTIEAINNAKEKTANNTGLKLIFAINYGGRAELVHSIKNMFDELHQQGLNSDIIDETYINNHLMTKDYPDPELLIRTSGEQRISNFLIWQVSYSEFIFNQKLWPDFDEDELIKCIKIYQSRQRRFGGLSEE

SEQ ID NO: 2 - protein, 6 aa, Staphylococcus aureus
  misc_feature (4)..(4): Xaa can be any naturally occurring amino acid
ENWXRP

SEQ ID NO: 3 - protein, 5 aa, Staphylococcus aureus
  misc_feature (4)..(4): Xaa can be any naturally occurring amino acid
DGNXR

SEQ ID NO: 4 - protein, 6 aa, Staphylococcus aureus
DGNGRW

SEQ ID NO: 5 - protein, 6 aa, Staphylococcus aureus
ENWSRP

SEQ ID NO: 6 - protein, 6 aa, Staphylococcus aureus
PRIKGH

SEQ ID NO: 7 - protein, 6 aa, Staphylococcus aureus
RYSNFL

SEQ ID NO: 8 - protein, 5 aa, Staphylococcus aureus
HIAII

SEQ ID NO: 9 - protein, 6 aa, Staphylococcus aureus
NGRWAK

SEQ ID NO: 10 - protein, 22 aa, Staphylococcus aureus
RIKGHYEGMQTIKKITRVASDI

SEQ ID NO: 11 - protein, 8 aa, Staphylococcus aureus
YLTLYAFS

SEQ ID NO: 12 - protein, 24 aa, Staphylococcus aureus
ESEVNYIMNLPVNFLKTFLPELIE

SEQ ID NO: 13 - protein, 5 aa, Staphylococcus aureus
KVETI

SEQ ID NO: 14 - protein, 14 aa, Staphylococcus aureus
KSTIEAINNAKEKT

SEQ ID NO: 15 - protein, 8 aa, Staphylococcus aureus
KLIFAINY

SEQ ID NO: 16 - protein, 17 aa, Staphylococcus aureus
GRAELVHSIKNMFDELH

SEQ ID NO: 17 - protein, 5 aa, Staphylococcus aureus
ETYIN

SEQ ID NO: 18 - protein, 4 aa, Staphylococcus aureus
LLIR

SEQ ID NO: 19 - protein, 4 aa, Staphylococcus aureus
EFIF

SEQ ID NO: 20 - protein, 5 aa, Staphylococcus aureus
EDELI

(19) United States
(12) Patent Application Publication
(10) Pub. No.: US 2005/0208639 A1
(43) Pub. Date: Sep. 22, 2005

(54) Crystal structure of staphylococcus undecaprenyl pyrophosphate synthase and uses thereof

(75) Inventors: Mark Ammirati, Stonington, CT (US); Jayvardhan Pandit, Mystic, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/688,167

(22) Filed: Oct. 17, 2003

Related U.S. Application Data
(60) Provisional application No. 60/419,952, filed on Oct. 21, 2002.

(51) Int. Cl. C12N 9/12; G06F 19/00; G01N 33/48; G01N 33/50

(52) U.S. Cl. 435/194; 702/19

(57) ABSTRACT

The invention is directed generally to the structure of prenyltransferases, particularly undecaprenyl pyrophosphate synthase, an enzyme important in bacterial cell wall synthesis. The invention relates to the crystal structure of undecaprenyl pyrophosphate synthase from Staphylococcus aureus and the interaction with a cofactor and ligands. The invention also relates to the structure of ligand and cofactor binding sites.

### REFERENCE TO RELATED APPLICATION

This application claims priority to U.S. Provisional Patent Application 60/419,952, filed on Oct. 21, 2002.

### FIELD OF THE INVENTION

The invention is directed generally to the crystal structure of enzymes. More particularly, the invention relates to the atomic structure of the substrate-binding sites of enzymes involved in the chain elongation of isoprenoid chains and the use of the structure in drug design.

### SEQUENCE LISTING REFERENCE

This application includes one computer sequence listing (SEQ. ID NOS. 1 to 20) appendix submitted on one computer diskette and the material on the computer diskette sequence listing (SEQ. ID NOS. 1 to 20) is incorporated-by-reference into the present application.

### BACKGROUND OF THE INVENTION

Prenyltransferases are enzymes important in lipid, peptidoglycan, and glycoprotein biosynthesis. These enzymes act on molecules having a five-carbon isoprenoid substrate. Prenyltransferases are classified into two major subgroups according to whether they catalyze the cis- or trans-isomerization of products in the prenyl chain elongation. E-type prenyltransferases catalyze trans-isomerization and z-type prenyltransferases catalyze cis-isomerization. Unlike the trans-type prenyltransferases, the cis-prenyltransferases are poorly categorized. In particular, little is known about the detailed molecular structure of the active site of cis-prenyltransferases. In consequence, inhibitors of the cis-prenyltransferases have been difficult to establish using a structure-based approach. This deficiency is particularly important because cis-prenyltransferases are involved in the biosynthesis of peptidoglycan in prokaryotes and that of glycoproteins in eukaryotes. Such pathways are crucial for survival of the organism.

Bacterial undecaprenyl pyrophosphate synthase (UPS), also known as undecaprenyl diphosphate synthase, is a z-type prenyltransferase that catalyzes the sequential condensation of eight molecules of isoprenyl pyrophosphate (IPP) with trans, trans-farnesyl pyrophosphate (FPP) to produce the 55-carbon molecule termed undecaprenyl pyrophosphate. Undecaprenyl pyrophosphate is released from the synthase and dephosphorylated to form undecaprenyl phosphate that serves as the essential carbohydrate and lipid carrier in bacterial cell wall and lipopolysaccharide biosynthesis. Undecaprenyl pyrophosphate synthase differs from other members of the prenyltransferase family in the product stereochemistry and product chain length.

Emerging resistance to currently used antibacterial agents has generated an urgent need for antibiotics acting by different mechanisms. Undecaprenyl pyrophosphate synthase exists ubiquitously in bacteria and plays an essential and critical roll in the cell wall biosynthesis pathway. Thus, undecaprenyl pyrophosphate synthase is essential for cell viability and provides a valid and unexploited molecular target for antibacterial drug discovery. In consequence, a structure-based approach to development of inhibitors could provide novel antibiotics.

Fujihashi et al., 2001, show atomic coordinates for a crystal of undecaprenyl pyrophosphate from M. luteus, in the absence of substrate or cofactor. Fujihashi et al., 2001, Crystal structure of cis-prenyl chain elongating enzyme, undecaprenyl diphosphate synthase, 98 Proc. Natl. Acad. Sci USA 4337. Some amino acid residues are not defined in the crystal structure.

Ko et al., 2001, show atomic coordinates for a crystal of undecaprenyl pyrophosphate from E. coli, in the absence of substrate or cofactor. Ko et al., 2001, Mechanism of Product Chain Length Determination and the Role of a Flexible Loop in E. coli Undecaprenyl-pyrophosphate Synthase Catalysis, 276 J. Biol. Chem. 47474. Some amino acid residues are not defined in the crystal structure.

Huang et al., U.S. Pat. No. 6,287,810 is directed to polynucleotides encoding an undecaprenyl pyrophosphate synthase of S. aureus, but does not teach a three-dimensional structure of undecaprenyl pyrophosphate synthase.

### SUMMARY OF THE INVENTION

The invention relates generally to protein crystal structures and uses thereof in drug design. More particularly, the invention relates to Staphylococcus undecaprenyl pyrophosphate synthase in crystalline form. The invention also relates to a composition comprising the synthase in crystalline form. The composition can further comprise at least one ligand.

In one embodiment, the invention comprises a composition comprising a Staphylococcus undecaprenyl pyrophosphate synthase in crystalline form, the synthase comprising an amino acid sequence at least about 80% homologous to SEQ ID NO:1. In a preferred embodiment of the synthase the amino acid sequence is at least about 90% homologous.

The synthase can have a first ligand binding site, a second ligand binding site, or both. Moreover, the composition comprising the synthase can comprise at least one ligand. The ligand can be co-crystallized with the synthase. Suitable ligands include, but are not limited to, farnesyl pyrophosphate, (S)-farnesyl thiopyrophosphate, isoprenyl phyrophosphate, magnesium ion, and sulfate ion. Preferably farnesyl pyrophosphate or (S)-farnesyl thiopyrophosphate are associated with the first ligand binding site and isoprenyl pyrophosphate or sulfate are associated with the second ligand binding site.

In another aspect the undecaprenyl pyrophosphate synthase comprises a first ligand binding site defined by at least one amino acid residue selected from the group consisting of Asp³³, Gly³⁴, Gly³⁶, Arg³⁷, Arg⁴⁶, Ala⁷⁶, Arg⁸⁴, Leu⁹⁵, Pro⁹⁶, and Phe¹⁴⁸. In a preferred embodiment, the crystal can comprise a first ligand binding site defined by amino acid residues 33, 34, 36, 37, 46, 76, 84, 95, 96, and 148 having atoms having atomic coordinates according to FIG. 5.

In yet another aspect, the undecaprenyl pyrophosphate synthase comprises a second binding site formed by at least one amino acid residue selected from the group consisting of Asp³³, Arg²⁰¹, Arg²⁰⁷, and Ser²⁰⁹ from one chain (A) of the dimer, and Glu²²⁰ and Gly²⁵ from the other chain (B) of the dimer. Thus, both polypeptide chains can contribute to the second binding site. In a preferred embodiment, the crystal can comprise a second ligand binding site defined by amino acid residues 33, 201, 207, 209, 220(B), and 251(B) having atoms having atomic coordinates according to FIG. 5.

The invention also relates to undecaprenyl pyrophosphate synthase in crystalline form wherein the synthase is S. aureus undecaprenyl pyrophosphate synthase.

According to the present invention, also provided is a selenomethionine substitution crystalline form of a Staphylococcus undecaprenyl pyrophosphate synthase. The synthase can be from S. aureus.

In still another aspect, the invention is directed to a composition comprising undecaprenyl pyrophosphate synthase in crystalline form and a substrate. The synthase can be from any organism, not limited to Staphylococcus.

One aspect of the invention is directed to a method of designing or identifying a potential ligand for an undecaprenyl pyrophosphate synthase comprising using a three-dimensional structure of an undecaprenyl pyrophosphate synthase, employing the three dimensional structure to design or select the potential ligand, obtaining the potential ligand; and contacting the potential ligand with the undecaprenyl pyrophosphate synthase to determine binding to the undecaprenyl pyrophosphate synthase. One skilled in the art will recognize that the steps of the method can be carried out in various orders. The three-dimensional structure of a binding site can be defined by atomic coordinates of amino acid residues 33, 34, 36, 46, 76, 84, 95, 96, and 148 according to FIG. 5.

The method can further comprise identifying chemical entities or fragments thereof, capable of binding to the undecaprenyl pyrophosphate synthase; and assembling the identified chemical entities or fragments thereof into a single molecule to provide the structure of the potential ligand.

The potential ligand can be an inhibitor. In one embodiment the inhibitor is a competitive inhibitor. In another embodiment the inhibitor is a non-competitive inhibitor. The ligand can be designed de novo. Alternatively, the ligand can be designed from a known inhibitor. The method can further comprise using the atomic coordinates according to FIG. 5, or portion thereof, of a ligand bound to the undecaprenyl pyrophosphate synthase.

Another aspect of the invention is directed to a method for identifying a potential inhibitor of a mutant undecaprenyl pyrophosphate synthase, the method comprising using a three-dimensional structure of undecaprenyl pyrophosphate synthase as defined by atomic coordinates of undecaprenyl pyrophosphate synthase according to FIG. 5; replacing one or more undecaprenyl pyrophosphate synthase amino acids selected from 33, 34, 36, 37, 46, 76, 84, 95, 96, 148, 201, 207, 209, 220, and 251 of SEQ ID NO:1 in the three-dimensional structure with a different naturally occurring amino acid, thereby forming a mutant undecaprenyl pyrophosphate synthase; employing the three-dimensional structure to design or select the potential inhibitor; synthesizing the potential inhibitor; and contacting the potential inhibitor with the mutant undecaprenyl pyrophosphate synthase or the undecaprenyl pyrophosphate synthase in the presence of a substrate to test the ability of the potential inhibitor to inhibit the undecaprenyl pyrophosphate synthase or the mutant undecaprenyl pyrophosphate synthase. The potential inhibitor can be selected from a database.

In another aspect, the invention is directed to a method for identifying a potential inhibitor for an undecaprenyl pyrophosphate synthase, comprising using a three-dimensional structure of the synthase as defined by atomic coordinates of undecaprenyl pyrophosphate synthase according to FIG. 5; employing said three-dimensional structure to design or select the potential inhibitor; synthesizing the potential inhibitor; and contacting the potential inhibitor with the synthase in the presence of a substrate to determine the ability of the potential inhibitor to inhibit the synthase.

In one embodiment, the three-dimensional structure can be further defined by atomic coordinates of amino acid residues 201, 207, and 209 according to FIG. 5. In another embodiment, the three-dimensional structure can be further defined by atomic coordinates of amino acid residues 220(B) and 251(B), according to FIG. 5. Amino acid residues labeled “B” are from the complementary polypeptide chain of the dimer.

The potential ligand can be designed to form a hydrogen bond with at least one amino acid residue selected from the group consisting of Gly³⁴, Gly³⁶, Arg³⁷, Arg⁴⁵, and Arg⁸⁴. In addition, or alternatively, the potential ligand can be designed to form a hydrogen bond with at least one amino acid residue selected from the group consisting of Arg²⁰¹, Arg²⁰⁷, Ser²⁰⁹, Glu²²⁰(B), and Gly²⁵¹(B). In another embodiment, the potential ligand can be designed to form a hydrophobic bond with at least one amino acid residue selected from the group consisting of Ala⁷⁶, Leu⁹⁵, Pro⁹⁶, and Phe¹⁴⁸.

In one aspect the invention is directed to a ligand identified by these methods.

The invention also relates to a method of identifying a ligand capable of binding to an undecaprenyl pyrophosphate synthase substrate binding site, comprising: (a) introducing into a suitable computer program information defining the binding site comprising first atomic coordinates of amino acids capable of binding to a synthase substrate, wherein the program displays the three-dimensional structure of the binding site; (b) creating a three dimensional model of a test compound in the computer program; (c) docking the model of the test compound to the structure of the binding site; (d) creating a second three dimensional model of the substrate or an inhibitor of the synthase and docking the second model thereto; and (e) comparing the docking of the test compound and of the substrate or an inhibitor of the synthase to provide an output of the program. In one embodiment, the method further comprises introducing into the computer program second atomic coordinates of water molecules bound to the substrate. In another embodiment, the method further comprising introducing into the computer program third atomic coordinates of at least one synthase structural element selected from the group consisting of an alpha helix, a 3₁₀ helix, a strand of beta sheet, and a coil. The 3₁₀ helix can comprise the amino acid residue sequence Asn Trp Ser.

In yet another embodiment the method further comprises: (f) incorporating the test compound into a biological or biochemical assay for synthase activity; and (g) determining whether the test compound inhibits synthase activity in the assay.

The invention is also directed to a method of drug design comprising using the atomic coordinates of an S. aureus undecaprenyl pyrophosphate synthase, or substantial portion thereof, having at least one ligand binding site, to computationally evaluate relative associations of chemical entities with the ligand binding site. The chemical entity can be an intermediate in a farnesyl pyrophosphate elongation reaction, or an analog thereof.

In another aspect the invention is directed to a method for solving a crystal form comprising using the atomic coordinates of S. aureus undecaprenyl pyrophosphate synthase crystal, or portions thereof, to solve a crystal form of a mutant, homolog or co-complex of the undecaprenyl pyrophosphate synthase by Molecular Replacement. The method can further comprise using the atomic coordinates of a ligand bound to undecaprenyl pyrophosphate synthase.

One aspect of the invention is directed to a machine-readable data storage medium comprising a data storage material encoded with machine-readable data comprising atomic coordinates comprising amino acid residues 33, 34, 36, 37, 46, 76, 84, 95, and 148 according to FIG. 5. In one embodiment, the machine-readable data further comprise atomic coordinates comprising at least one amino acid residue selected from the group consisting of 201, 207, 209, 220(B), and 251(B) according to FIG. 5. In another embodiment, the machine-readable data comprise the three-dimensional structure of S. aureus undecaprenyl pyrophosphate synthase.

In another aspect, the invention comprises a computer-implemented tool for design of a drug, comprising: (a) a three-dimensional structure of a undecaprenyl pyrophosphate synthase as defined by atomic coordinates of a S. aureus undecaprenyl pyrophosphate synthase having at least one ligand binding site; (b) a model of a chemical entity; and (c) a computer program addressing the coordinates and capable of modeling the chemical entity in the ligand binding site to produce an output.

In yet another aspect, the invention comprises a computer for producing a three-dimensional representation of a undecaprenyl pyrophosphate synthase ligand binding site comprising: (a) a machine-readable data storage medium comprising a data storage material encoded with machine-readable data comprising the atomic coordinates comprising the amino acid residues 33, 34, 36, 37, 46, 76, 84, 95, and 148 according to FIG. 5; (b) a working memory for storing instructions for processing the machine-readable data; (c) a central-processing unit coupled to the working memory and to the machine-readable data storage medium for processing the machine readable data into the three-dimensional representation; and (d) a display coupled to the central-processing unit for displaying the three-dimensional representation. The computer can also produce a three-dimensional representation of the ligand binding site of an undecaprenyl pyrophosphate synthase; and the machine-readable data can comprise the atomic coordinates of the ligand binding site.

### BRIEF DESCRIPTION OF THE FIGURES

FIG. 1 is a topology diagram of S. aureus undecaprenyl pyrophosphate synthase.

FIGS. 2a and 2b are ribbon diagrams of the crystal structure of S. aureus undecaprenyl pyrophosphate synthase. Two orthogonal views of the dimer are shown in FIGS. 2a and 2b. In each figure, helices are labeled H and beta strands S. The ligands, FPP, Mg and Sulfate, are also labeled.

FIG. 3 is a ball and stick diagram of a part of the active site of S. aureus undecaprenyl pyrophosphate synthase showing all the interactions between protein and bound FPP and Mg.

FIG. 4 is a ball and stick diagram of another part of the active site S. aureus undecaprenyl pyrophosphate synthase showing all the interactions between protein and bound sulfate.

FIG. 5 shows the atomic coordinates of the polypeptide chains of S. aureus undecaprenyl pyrophosphate synthase.

### DETAILED DESCRIPTION OF THE INVENTION

In order that the invention described herein may be more fully understood, the following detailed description is set forth. The following table lists the amino acid abbreviations used herein.

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

The following terms are used herein as follows, unless stated otherwise:

The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine. Unless specifically indicated, all amino acids referred to in this application are in the L-form.

The term “unnatural amino acids” means amino acids that are not naturally found in proteins. Examples of unnatural amino acids used herein, include selenocysteine and selenomethionine. In addition, unnatural amino acids include D-phenylalanine and the D or L forms of nor-leucine, para-nitrophenylalanine, homophenylalanine, para-fluorophenylalanine, 3-amino-2-benzylpropionic acid, and homoarginine.

The term “positively charged amino acid” includes any naturally occurring or unnatural amino acid having a positively charged side chain under normal physiological conditions. Examples of positively charged naturally occurring amino acids are arginine, lysine and histidine.

The term “negatively charged amino acid” includes any naturally occurring or unnatural amino acid having a negatively charged side chain under normal physiological conditions. Examples of negatively charged naturally occurring amino acids are aspartic acid and glutamic acid.

The term “hydrophobic amino acid” means any amino acid having an uncharged, nonpolar side chain that is relatively insoluble in water. Examples of naturally occurring hydrophobic amino acids are alanine, leucine, isoleucine, valine, proline, phenylalanine, tryptophan and methionine. Histidine and tyrosine can also participate in hydrophobic bonds.

The term “hydrophilic amino acid” means any amino acid having an uncharged, polar side chain that is relatively soluble in water. Examples of naturally occurring hydrophilic amino acids are serine, threonine, tyrosine, asparagine, glutamine, and cysteine.

The term “hydrogen bond” is used to describe an interaction between polar atoms including N, O, and S, in which hydrogen forms a bridge. The side chains of ionic and hydrophilic amino acids and of amide moieties in the peptide backbone are candidates for hydrogen bonds. Polar and ionic moieties in substrates and inhibitors are candidates for hydrogen bonding.

The term “hydrophobic bond” is used to describe a Van der Waals interaction of non-polar moieties that are enthalpicly or entropicly favored over interaction with water or polar groups. Thus, one model for hydrophobic bonds is the gain in free energy formed by exclusion of water. Prime candidates for forming hydrophobic bonds are the aliphatic tail of farnesyl pyrophosphate and side chains of amino acid residues including phenylalanine, tryptophan, proline, leucine, isoleucine, valine, alanine, histidine, and tyrosine.

The term “residue” in amino acid residue refers to the part of an amino acid incorporated into a polypeptide.

The term “ligand” refers to a chemical entity that binds to, or associates with, a synthase. Often, but not always, a ligand is a small molecule. A substrate is a ligand that can be, under appropriate conditions, chemically acted upon by the synthase. In particular, farnesyl pyrophosphate is a substrate that binds to the synthase in the presence of magnesium ion, acting as a cofactor, but does not undergo a chemical reaction unless a second substrate, that is isoprenyl pyrophosphate, is present, and other conditions necessary for catalysis are met.

The term “mutant” refers to an undecaprenyl pyrophosphate synthase polypeptide, i.e. a polypeptide displaying the biological activity of wild-type, undecaprenyl pyrophosphate synthase, characterized by the replacement of at least one amino acid from the wild-type, undecaprenyl pyrophosphate synthase sequence according to SEQ ID NO:1. Such a mutant may be prepared, for example, by expression of undecaprenyl pyrophosphate synthase cDNA previously altered in its coding sequence by oligonucleotide-directed mutagenesis, or other means well-known in the art.

Undecaprenyl pyrophosphate synthase mutants may also be generated by site-specific incorporation of unnatural amino acids into undecaprenyl pyrophosphate synthase proteins using the general biosynthetic method of Noren, C. J., et al., Science, 244, pp. 182-188 (1989). In this method, the codon encoding the amino acid of interest in wild-type undecaprenyl pyrophosphate synthase is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant undecaprenyl pyrophosphate synthase enzyme with the site-specific incorporated unnatural amino acid.

Selenocysteine or selenomethionine may be incorporated into wild-type or mutant undecaprenyl pyrophosphate synthase as described below. In this method, the wild-type or mutagenized undecaprenyl pyrophosphate synthase cDNA may be expressed in a host organism on a growth medium depleted of either natural cysteine or methionine (or both) but enriched in selenocysteine or selenomethionine (or both).

Altered surface charge describes a change in one or more of the charge units of a mutant polypeptide, at physiological pH, as compared to wild-type undecaprenyl pyrophosphate synthase. This is preferably achieved by mutation of at least one amino acid of wild-type undecaprenyl pyrophosphate synthase to an amino acid comprising a side chain with a different charge at physiological pH than the original wild-type side chain.

The change in surface charge is determined by measuring the isoelectric point (pI) of the polypeptide molecule containing the substituted amino acid and comparing it to the isoelectric point of the wild-type undecaprenyl pyrophosphate synthase molecule.

Altered substrate specificity refers to a change in the ability of a mutant undecaprenyl pyrophosphate synthase to bind and use analogs of FPP, IPP, or both.

A “competitive” inhibitor is one that inhibits undecaprenyl pyrophosphate synthase activity by binding to the same form of undecaprenyl pyrophosphate synthase as its substrate binds—thus directly competing with the substrate for the active site of undecaprenyl pyrophosphate synthase. Competitive inhibition can be reversed completely by sufficiently increasing the substrate concentration.

An “uncompetitive” inhibitor is one that inhibits undecaprenyl pyrophosphate synthase by binding to a different form of the enzyme than does the substrate. Such inhibitors bind to undecaprenyl pyrophosphate synthase already bound with the substrate and not to the free enzyme. Uncompetitive inhibition cannot be reversed completely by increasing the substrate concentration.

A “non-competitive” inhibitor is one that can bind to either the free or substrate bound form of undecaprenyl pyrophosphate synthase.

Those of skill in the art may identify inhibitors as competitive, uncompetitive or non-competitive by computer fitting enzyme kinetic data using standard equations according to Segel, I. H., Enzyme Kinetics, J. Wiley & Sons, (1975).

The term “homologue” as used herein means a protein, polypeptide, oligopeptide or portion thereof, having preferably at least 80%, more preferably at least 90% amino acid sequence identity with Staphylococcus undecaprenyl pyrophosphate synthase or any functional or structural domain of undecaprenyl pyrophosphate synthase.

The term “co-complex” means undecaprenyl pyrophosphate synthase or a mutant or homologue of undecaprenyl pyrophosphate synthase in covalent or non-covalent association with a chemical entity or compound.

The term “associating with” refers to a condition of proximity between a chemical entity or compound, or portions thereof, and an undecaprenyl pyrophosphate synthase molecule or portions thereof. The association may be non-covalent, wherein the juxtaposition is energetically favored by hydrogen bonding or van der Waals or electrostatic interactions, or it may be covalent.

The terms “beta sheet or β-sheet” refers to the conformation of a polypeptide chain stretched into an extended zig-zig conformation. Portions of polypeptide chains termed strands that run “parallel” all run in the same direction, amino terminus to carboxy terminus. Polypeptide chains or portions thereof, termed strands, that are “antiparallel” run in the opposite directions.

The term “binding site” refers to a region of the synthase comprised of amino acid residues and optionally cofactors to which a ligand can bind. Undecaprenyl pyrophosphate synthase has binding sites for at least farnesyl pyrophosphate and longer chain derivatives of FPP, isoprenyl pyrophosphate, magnesium ion, and sulfate ion.

The term “active site” refers to any or all of the following sites in undecaprenyl pyrophosphate synthase: the FPP binding site, the IPP binding site, the site of the synthase reaction products and intermediates, the magnesium ion site, and the sulfate site. In one particular usage, “active site” refers to the site where the catalytic reaction occurs.

The term “atomic coordinates” refers to mathematical coordinates derived from mathematical equations related to the patterns obtained on diffraction of a monochromatic beam of X-rays by the atoms (scattering centers) of an undecaprenyl pyrophosphate synthase molecule in crystal form. The diffraction data are used to calculate an electron density map of the repeating unit of the crystal. The electron density maps are used to establish the positions of the individual atoms within the unit cell of the crystal. The similar term “structure coordinates” refers to the mathematical coordinates of the individual atoms.

The term “substantial portion” of atomic coordinates refers to a plurality of at least twelve atomic coordinates that define or partially define the location of several atoms in the synthase or ligand. Preferably, a substantial portion is at least 24 coordinates. More preferably, a substantial portion is at least 36 coordinates. The coordinates can be within the standard deviation.

The term “heavy atom derivatization” refers to a method of producing a chemically modified form of a crystal of undecaprenyl pyrophosphate synthase. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the enzyme. Blundel, T. L. and N. L. Johnson, Protein Crystallography, Academic Press (1976).

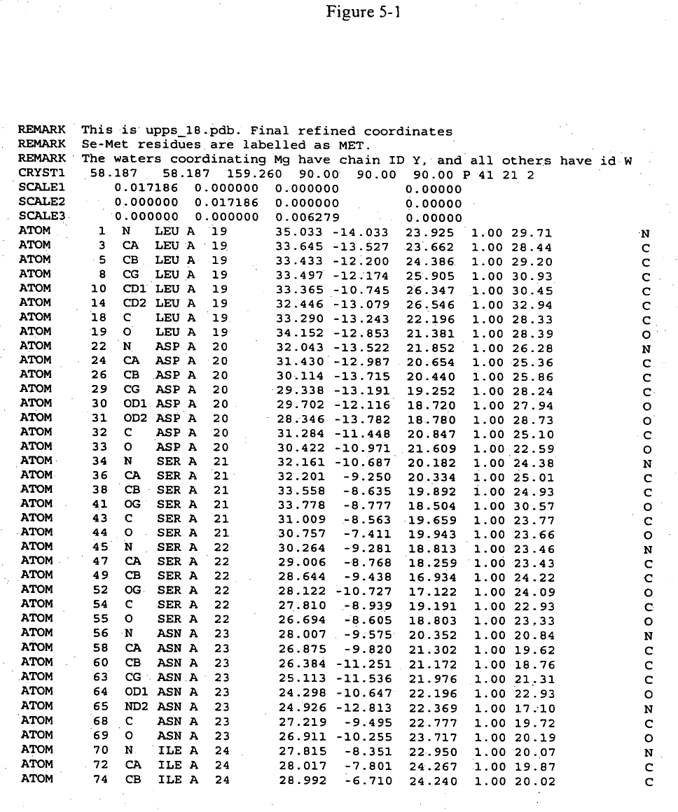

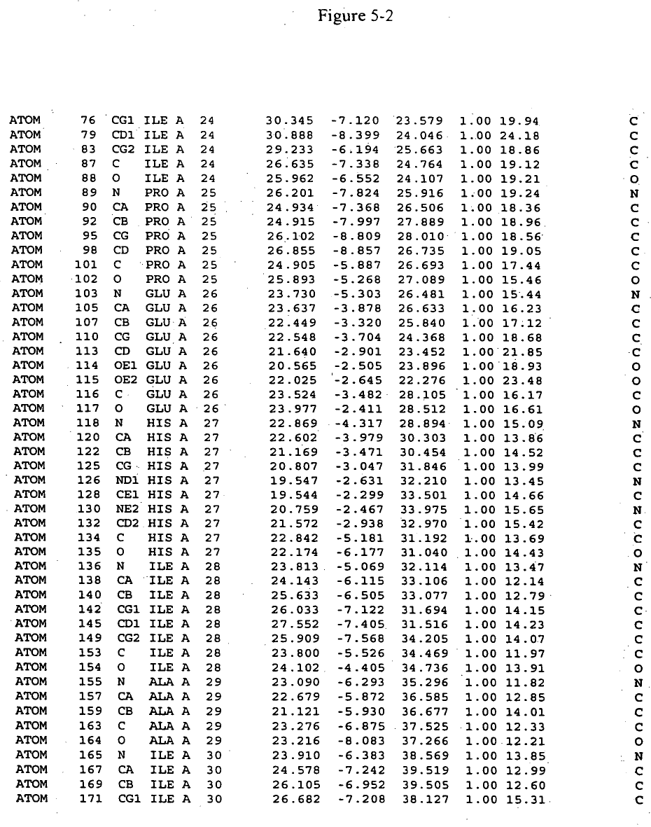

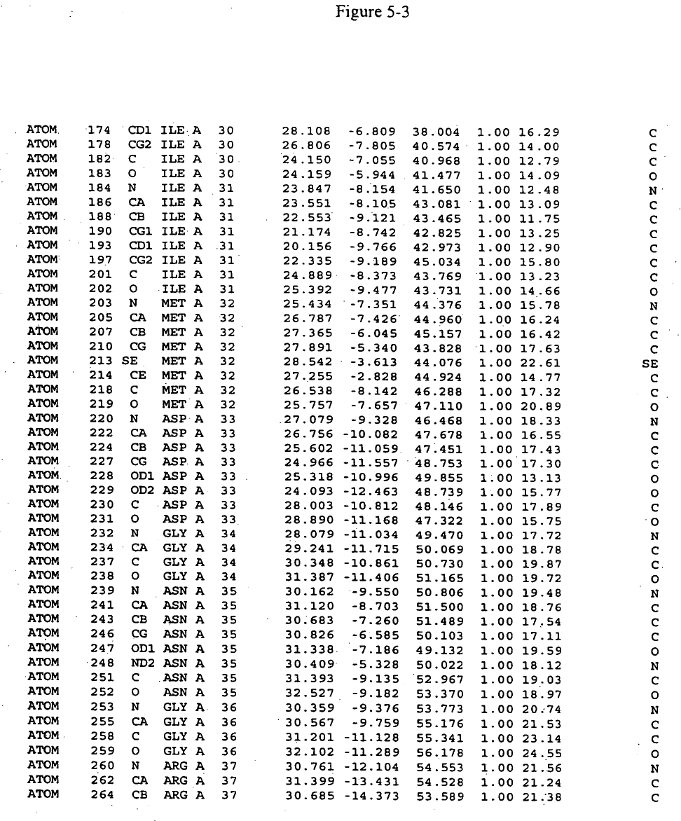

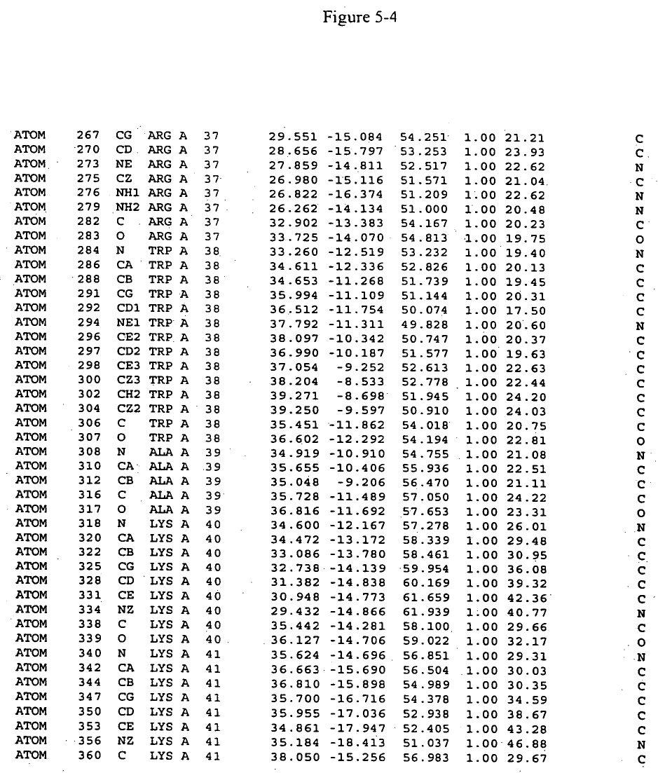

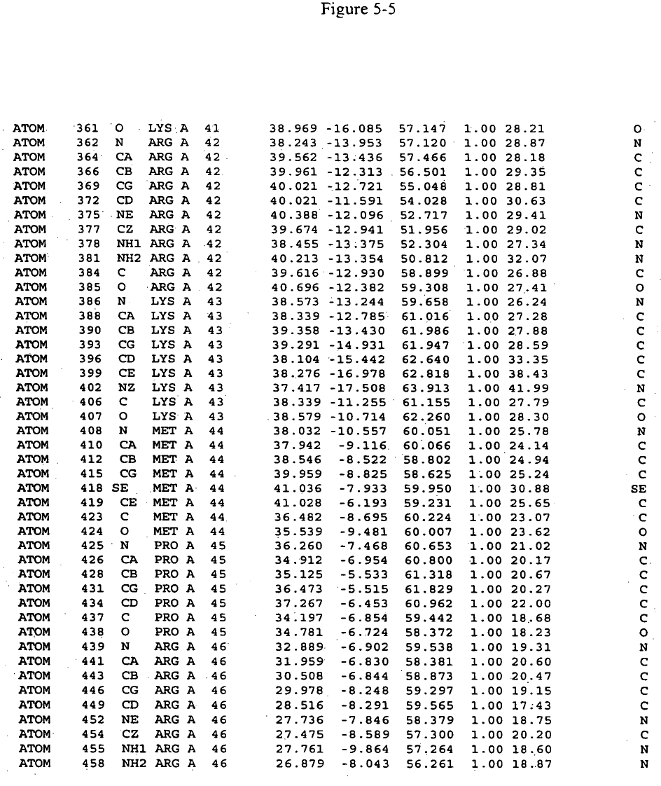

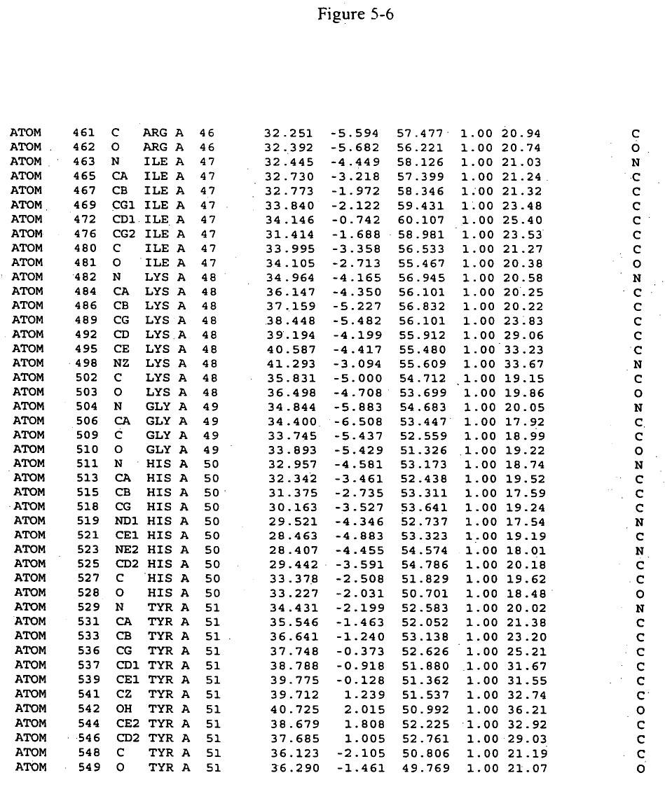

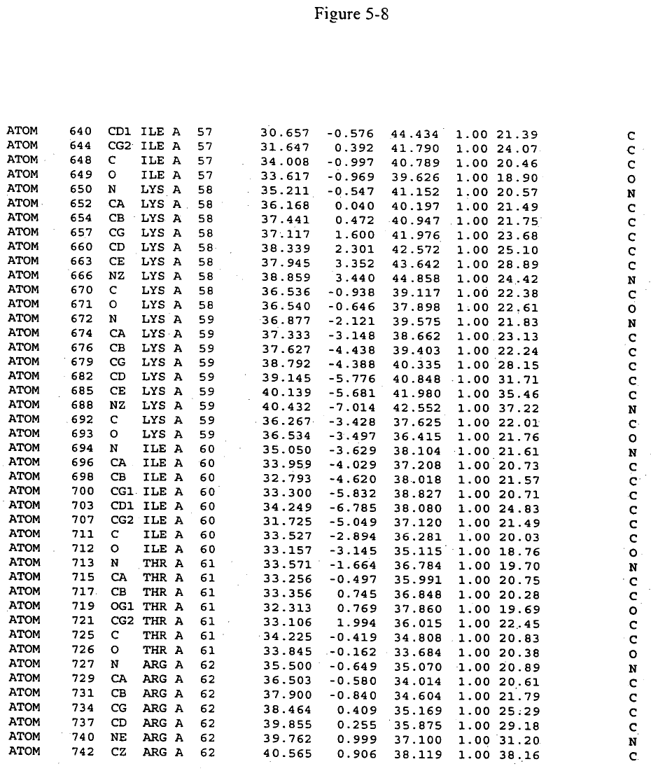

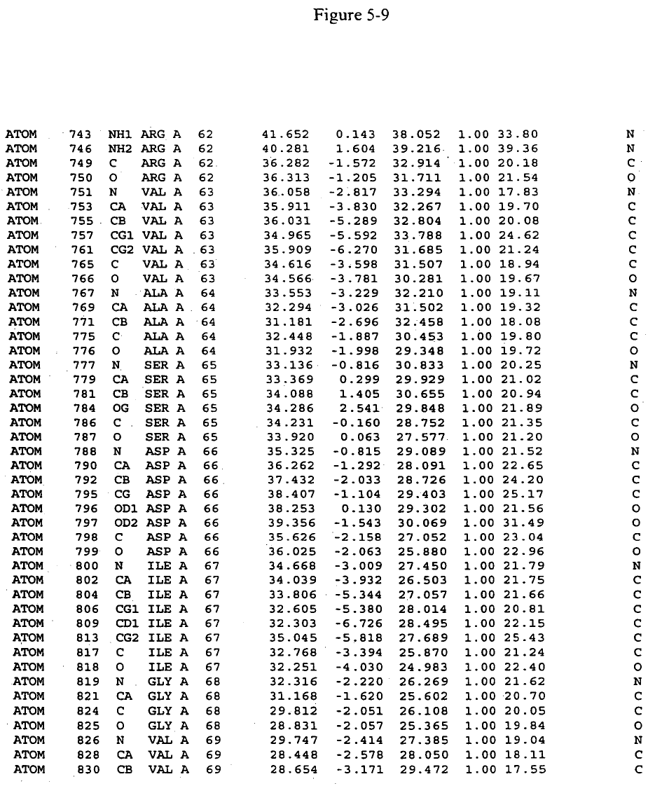

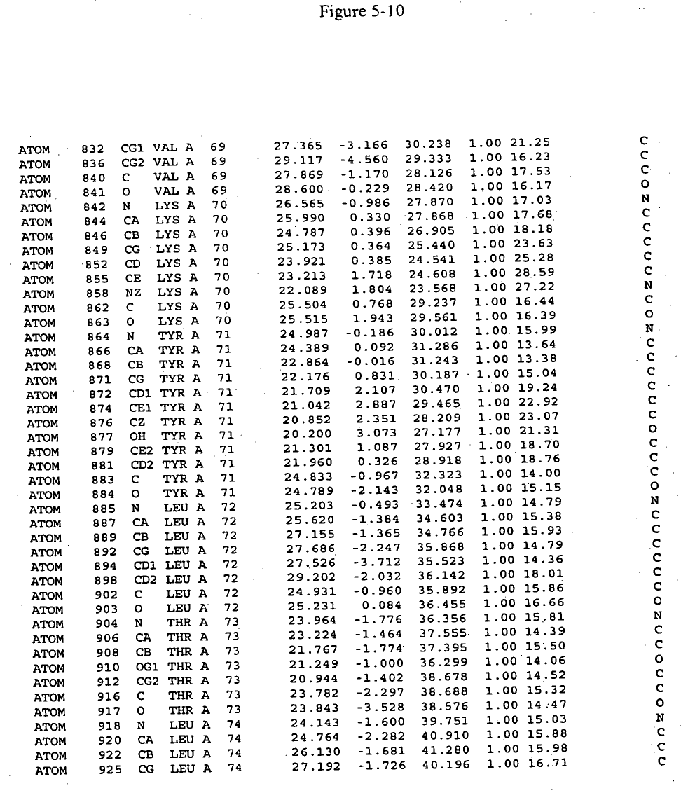

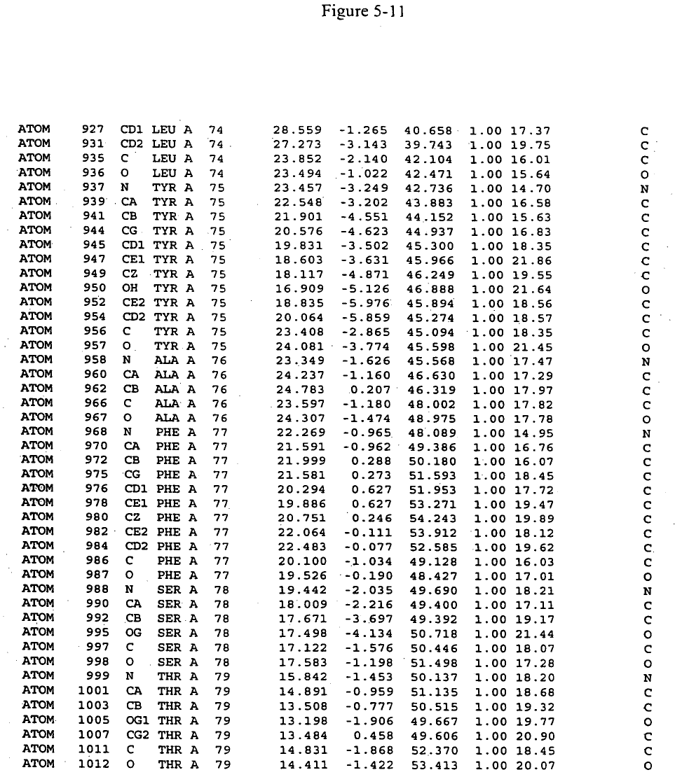

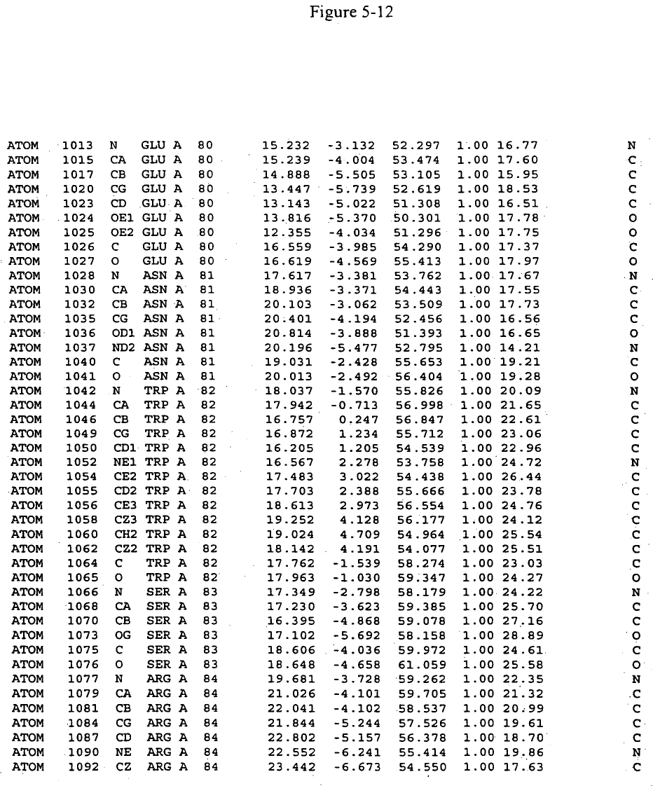

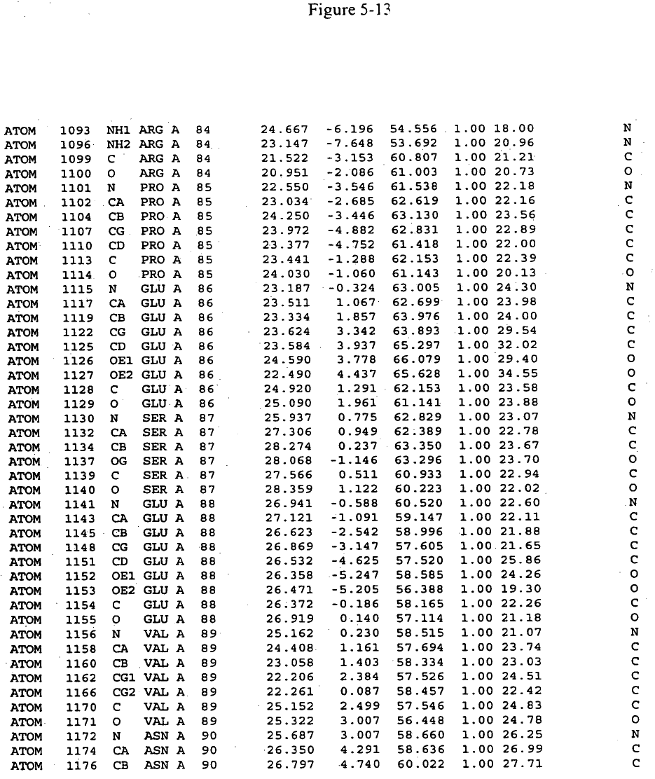

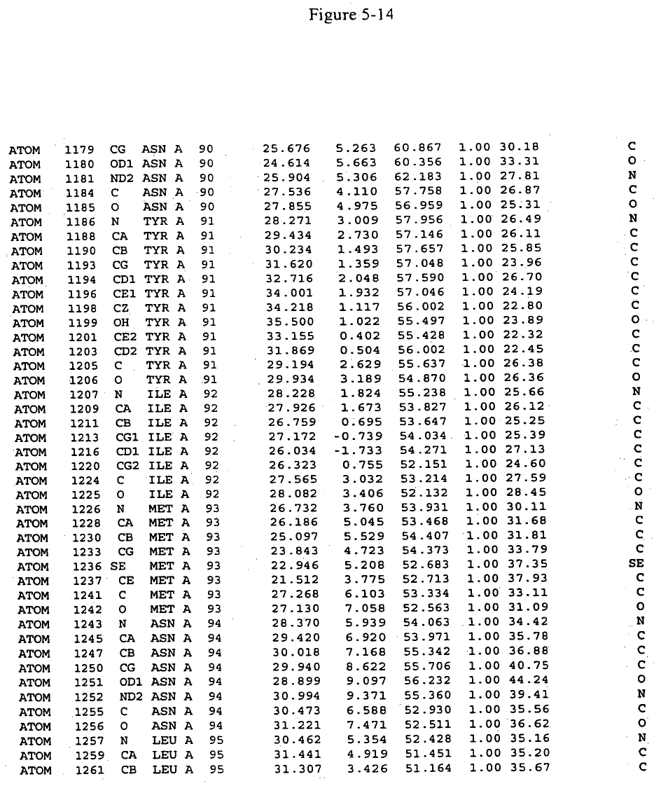

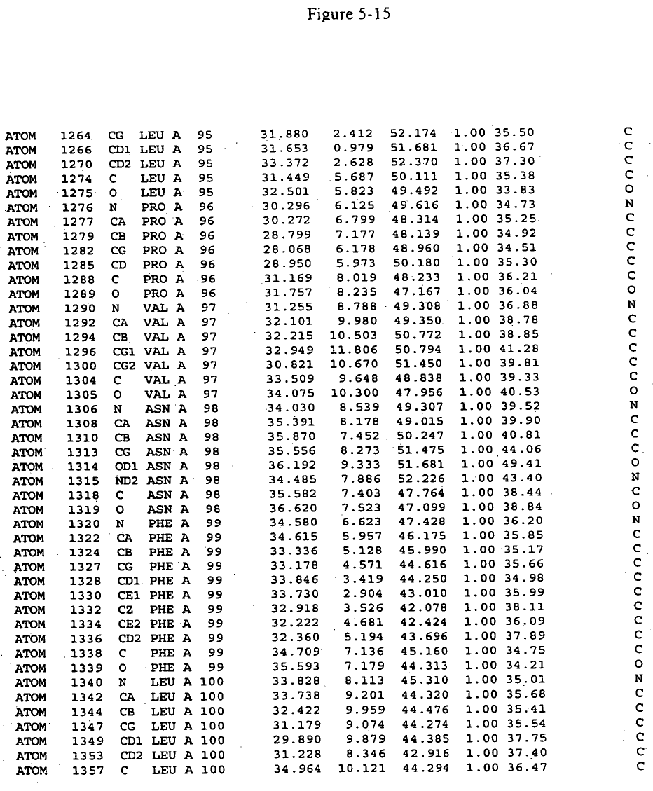

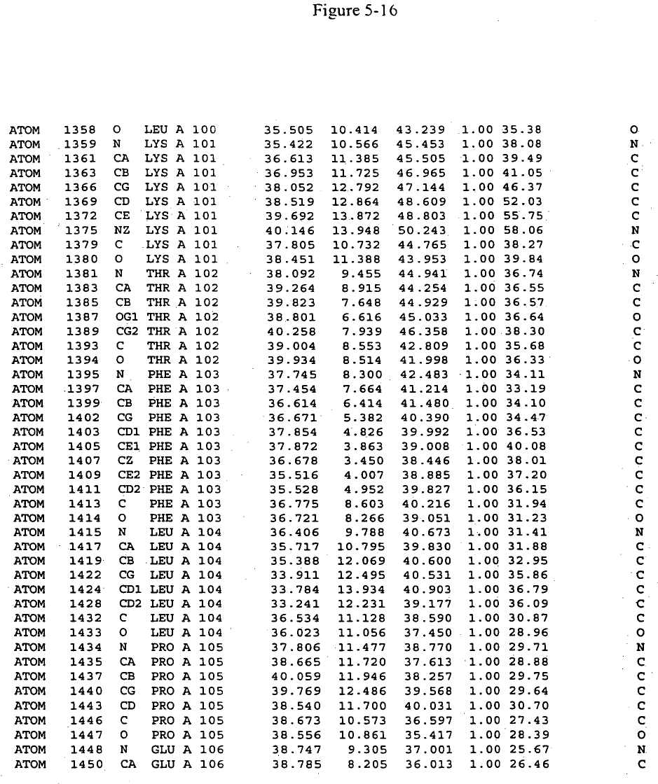

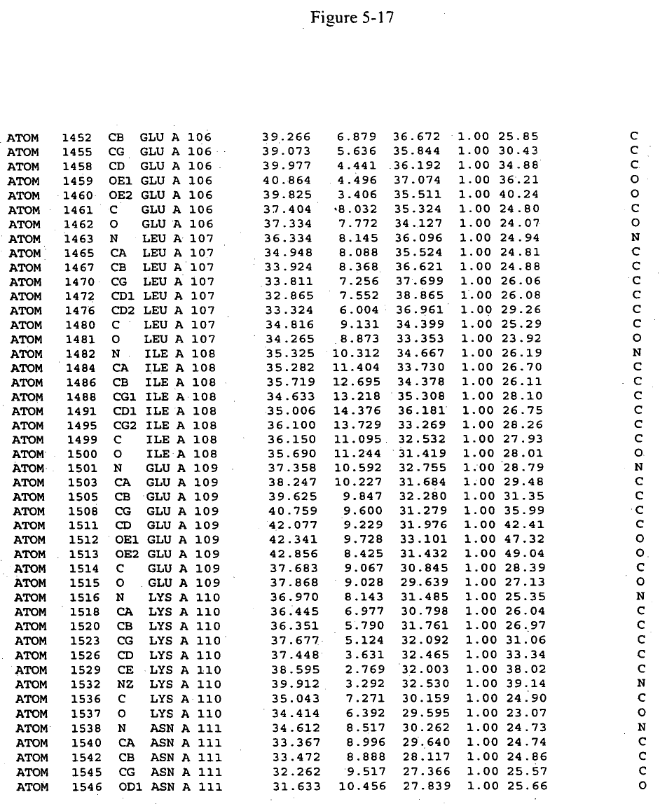

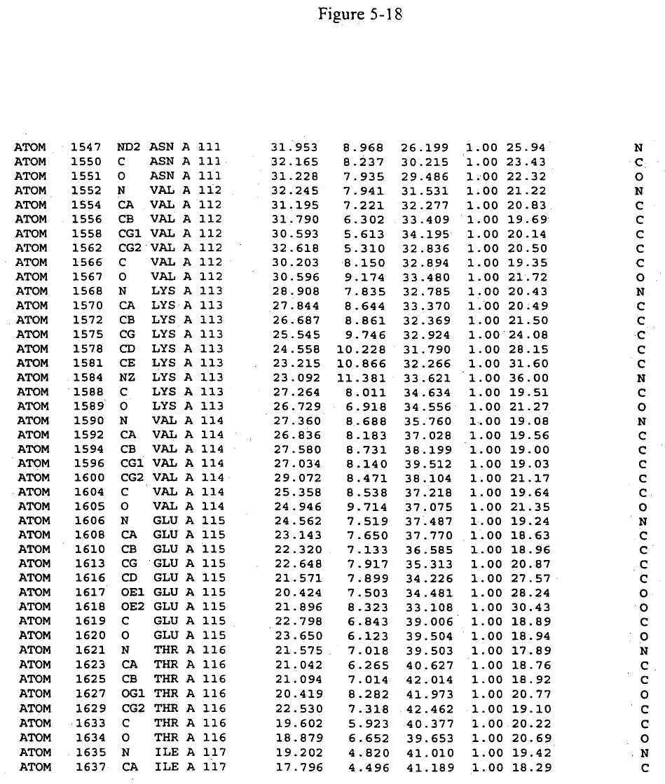

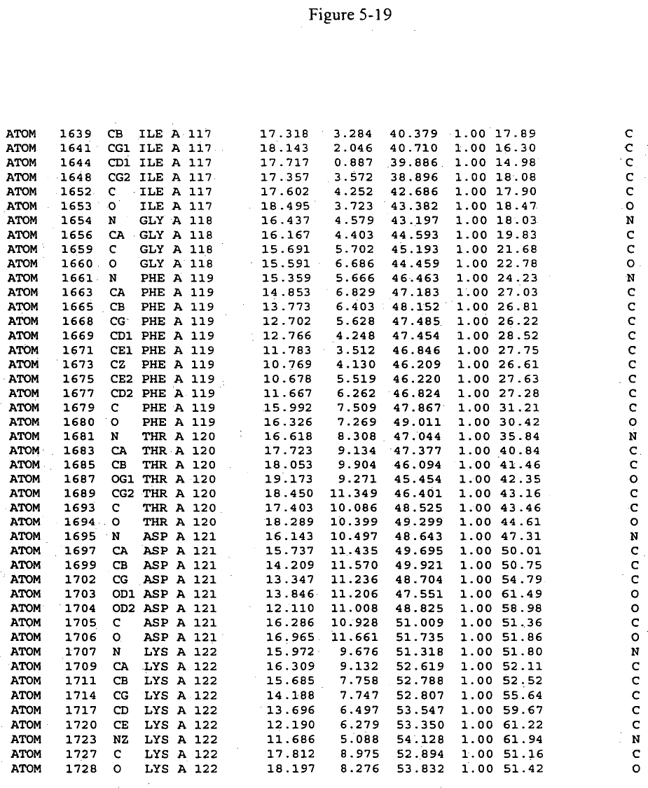

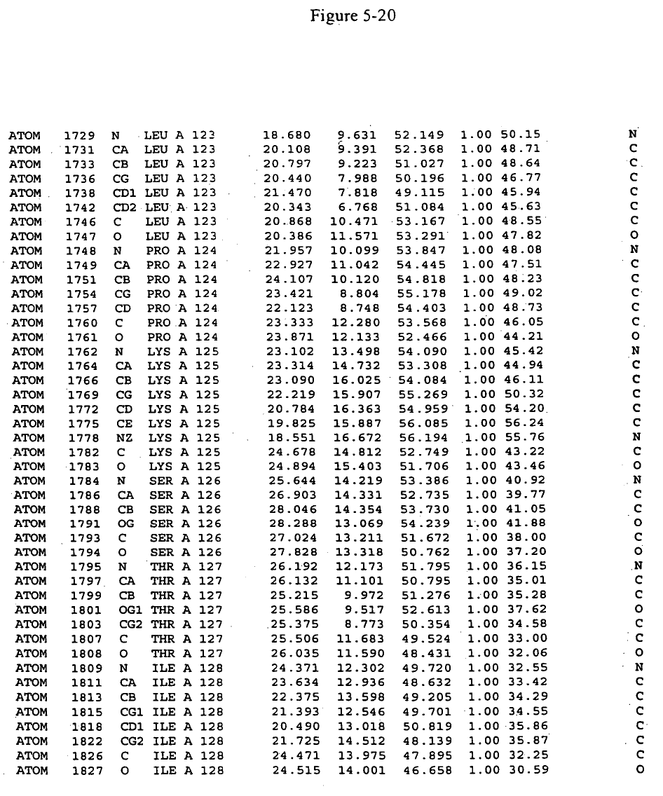

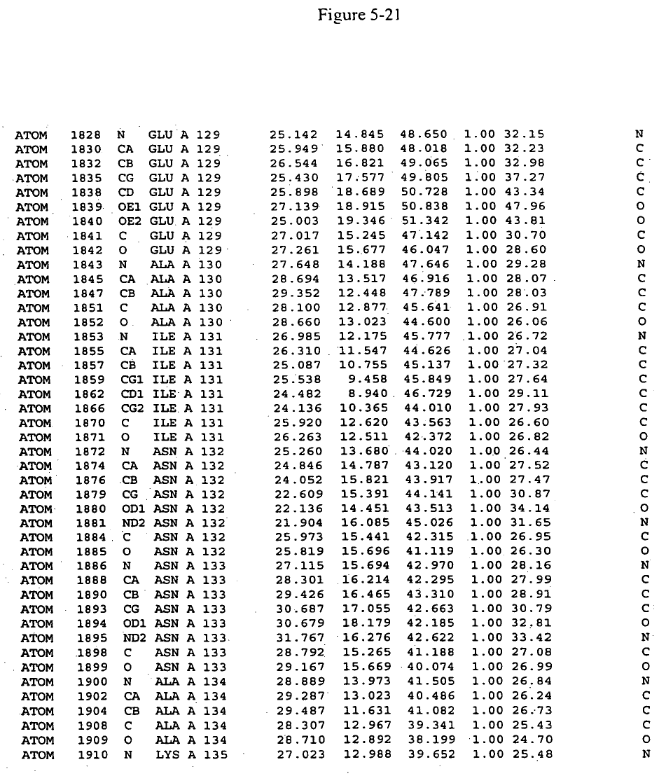

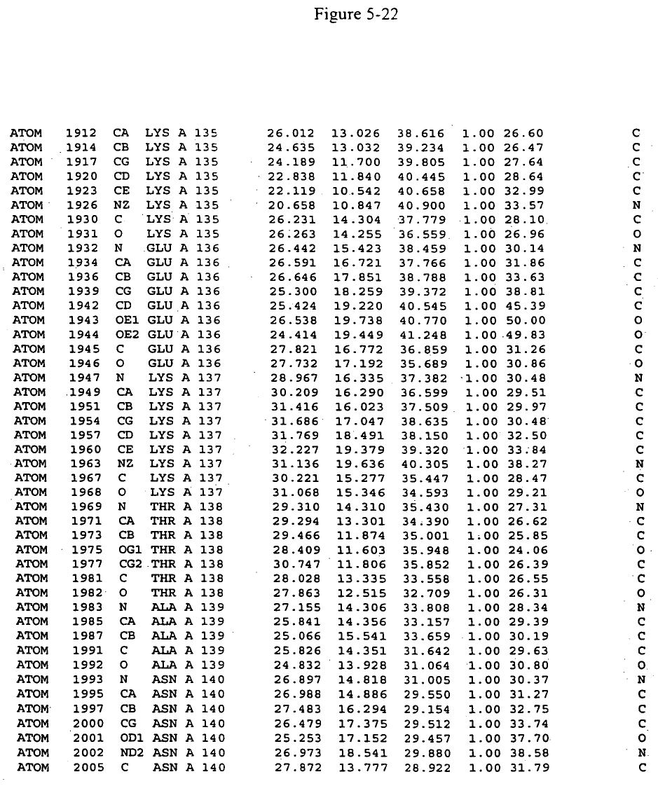

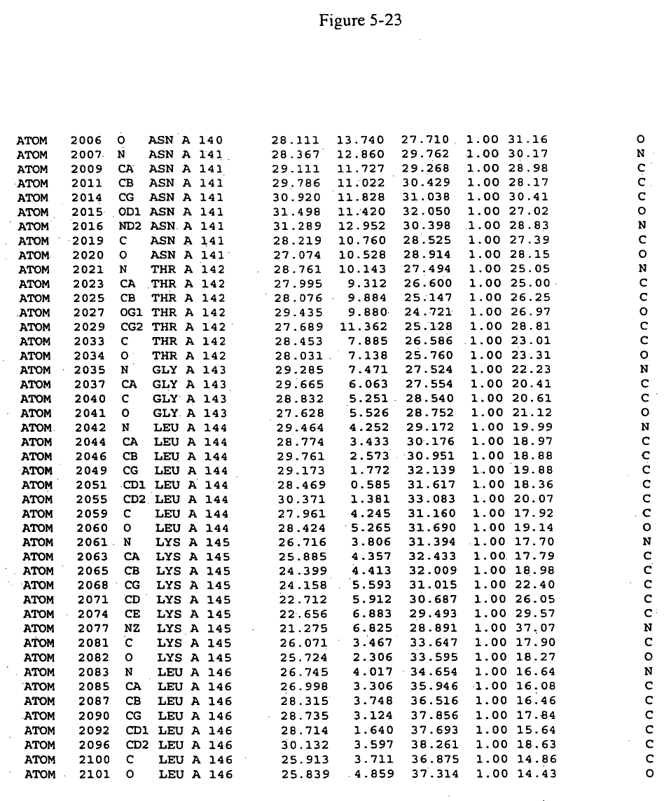

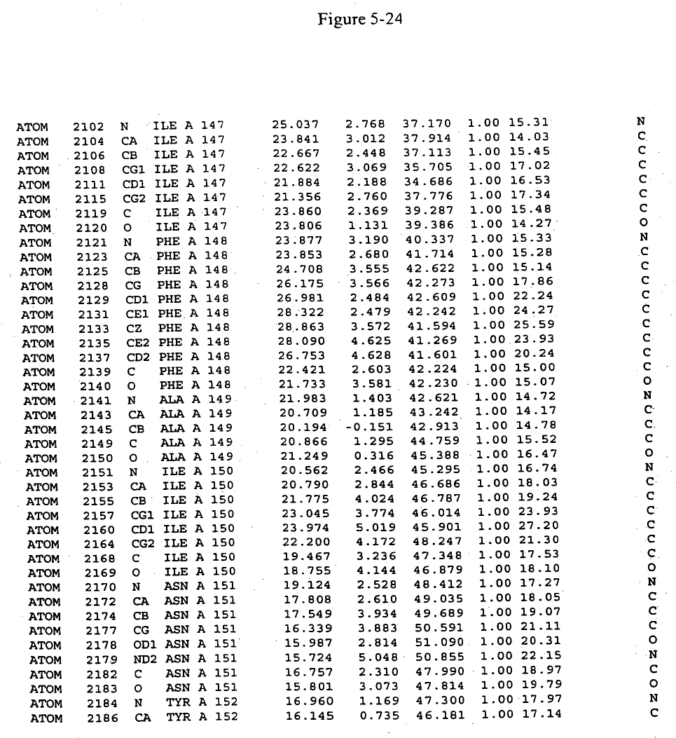

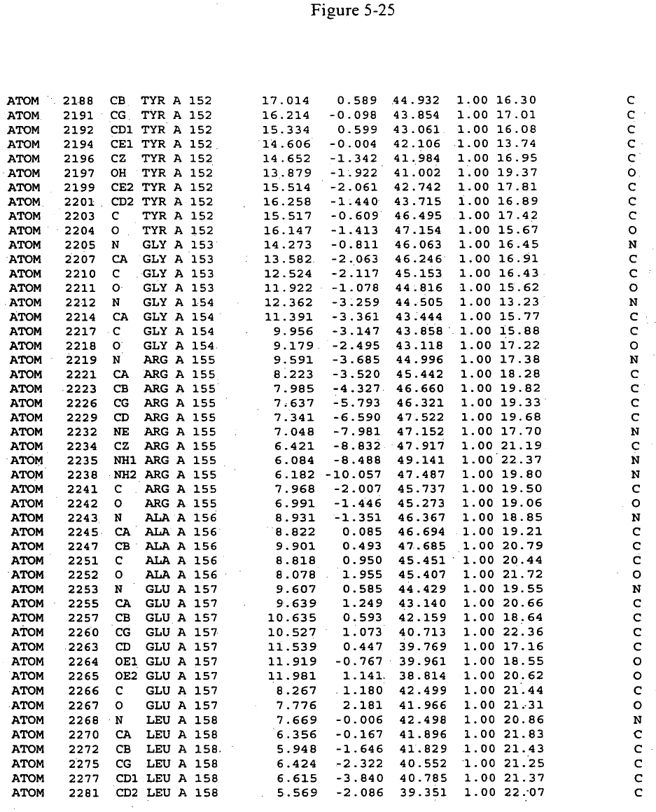

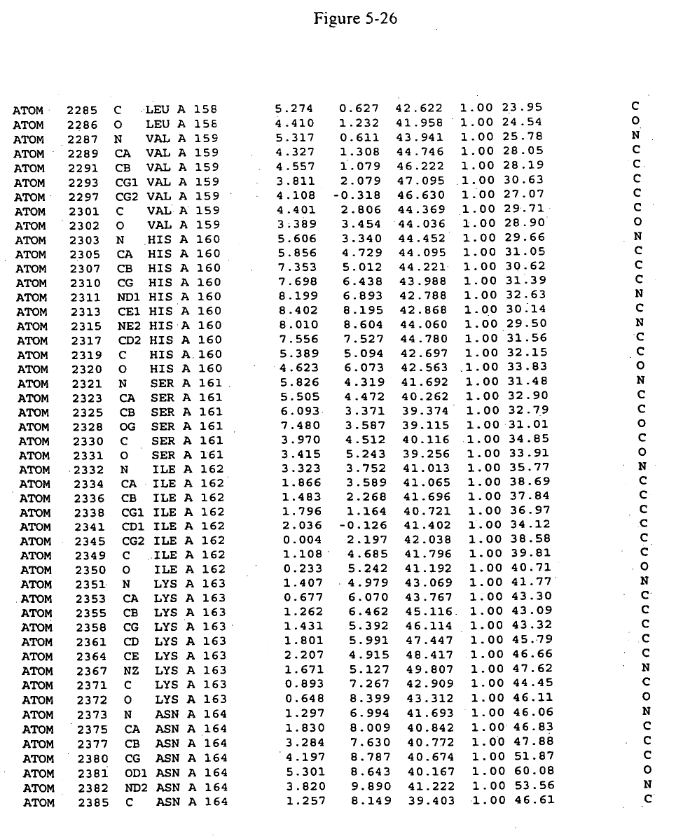

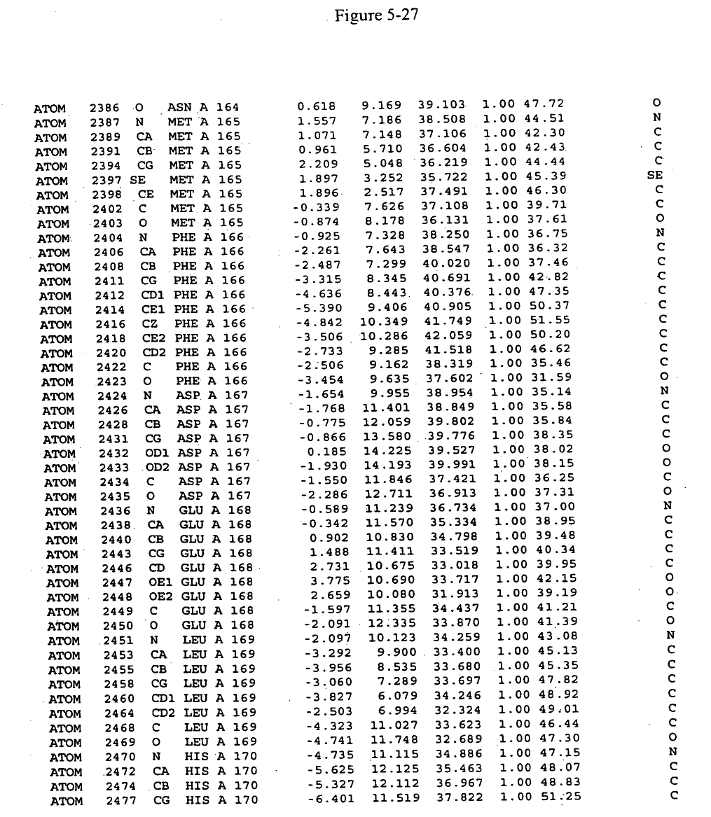

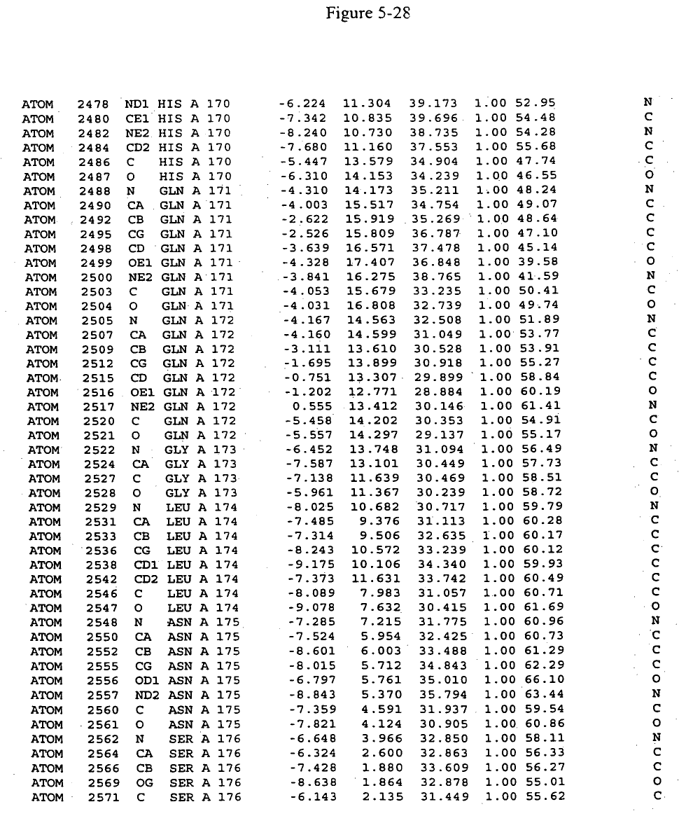

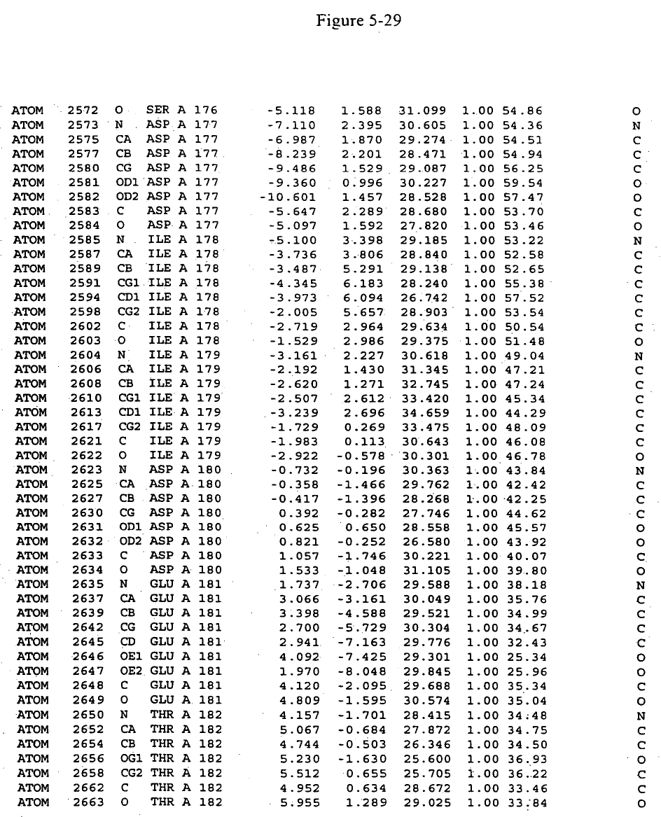

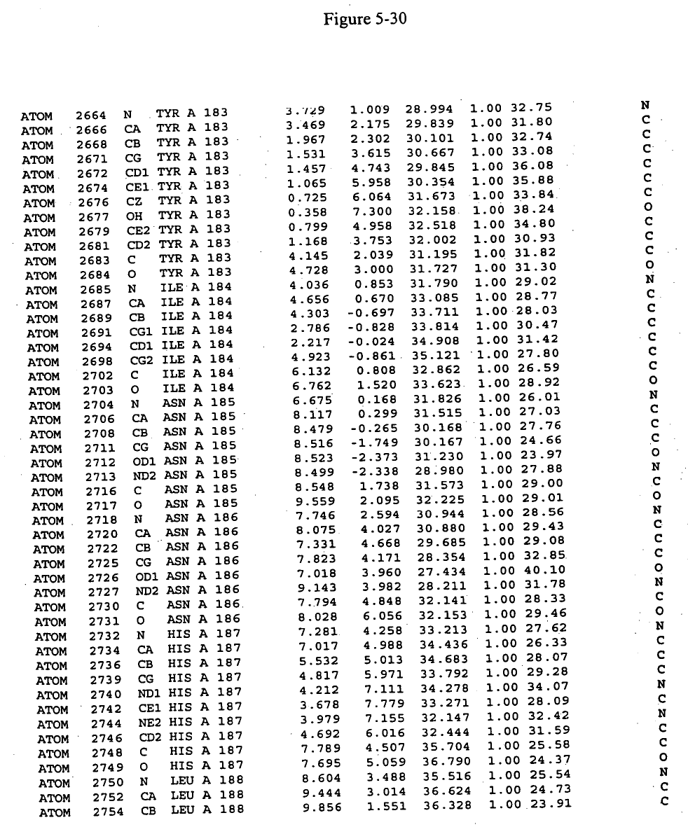

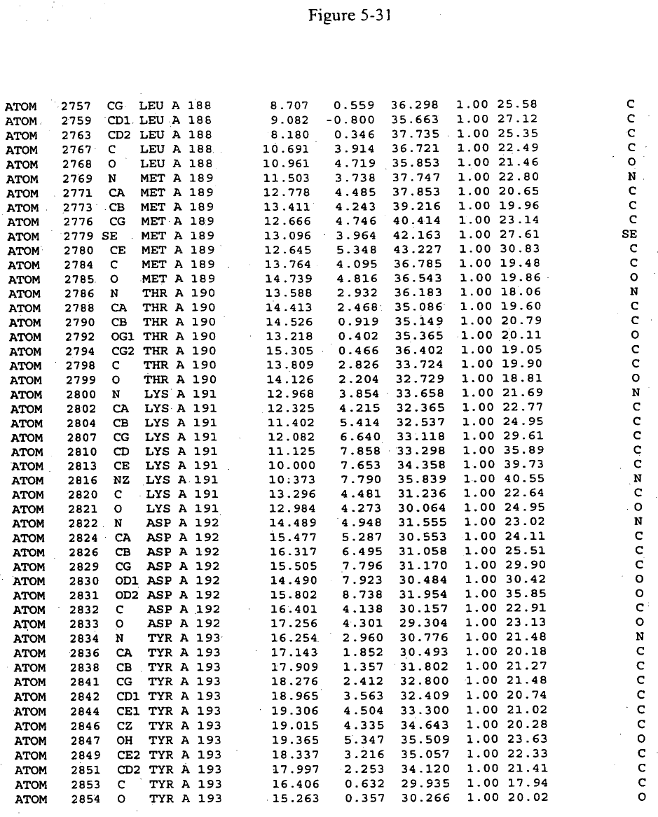

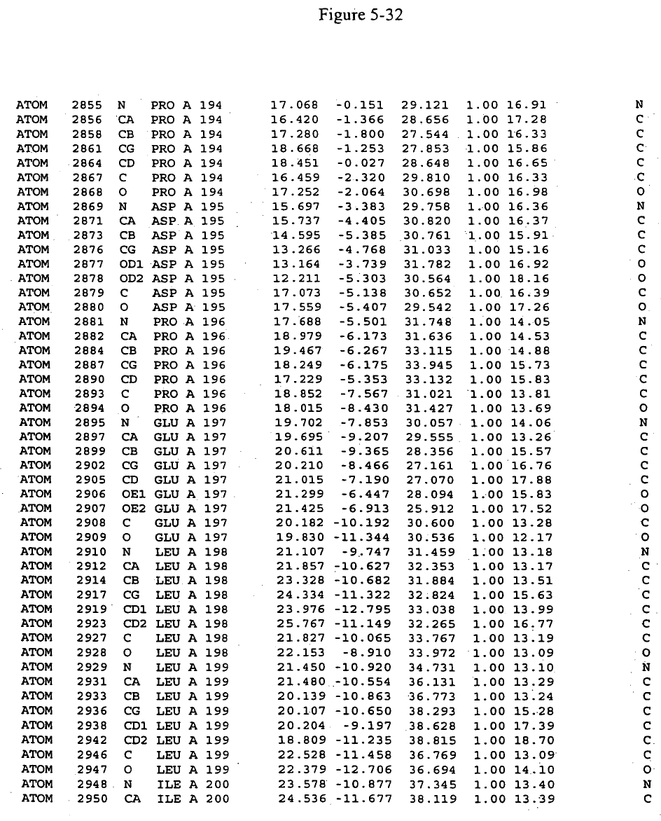

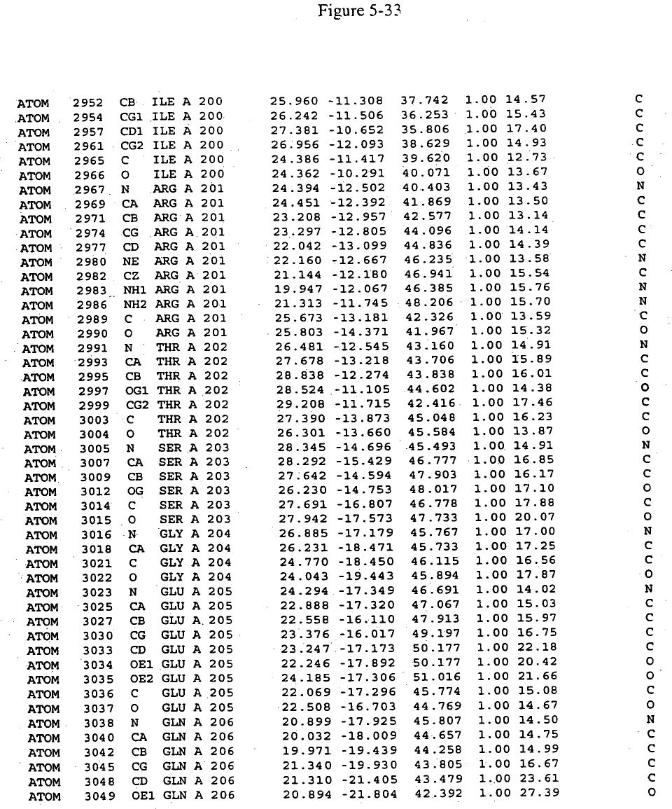

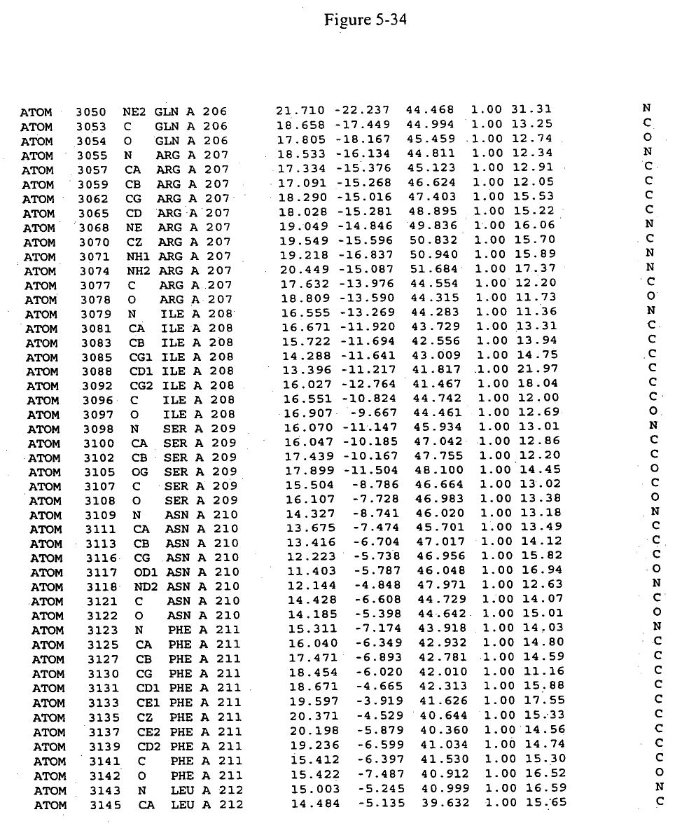

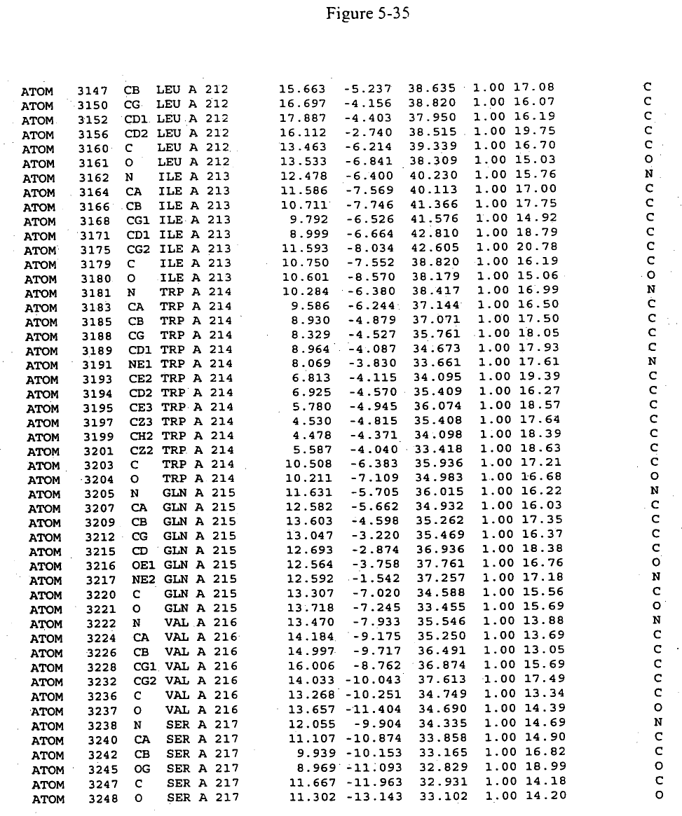

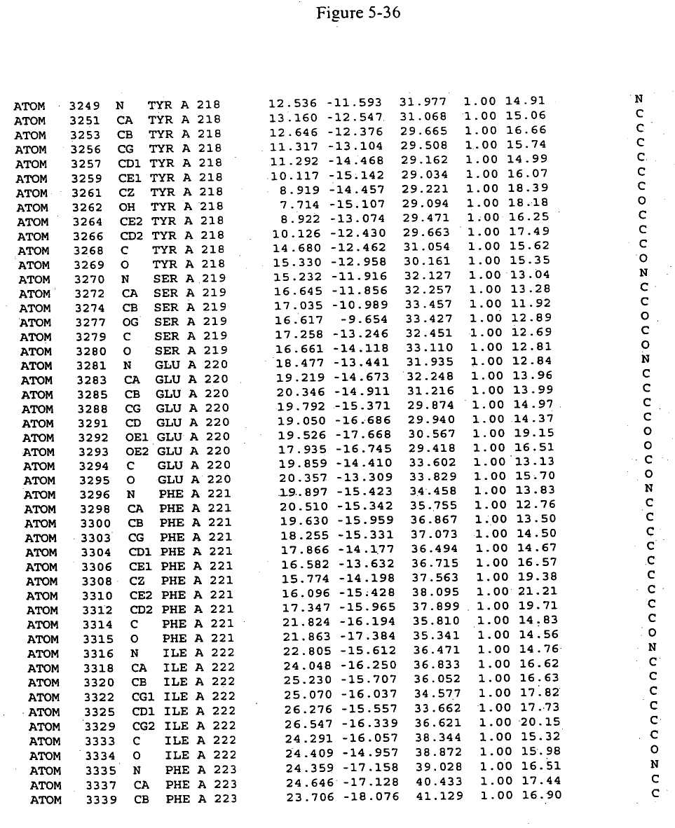

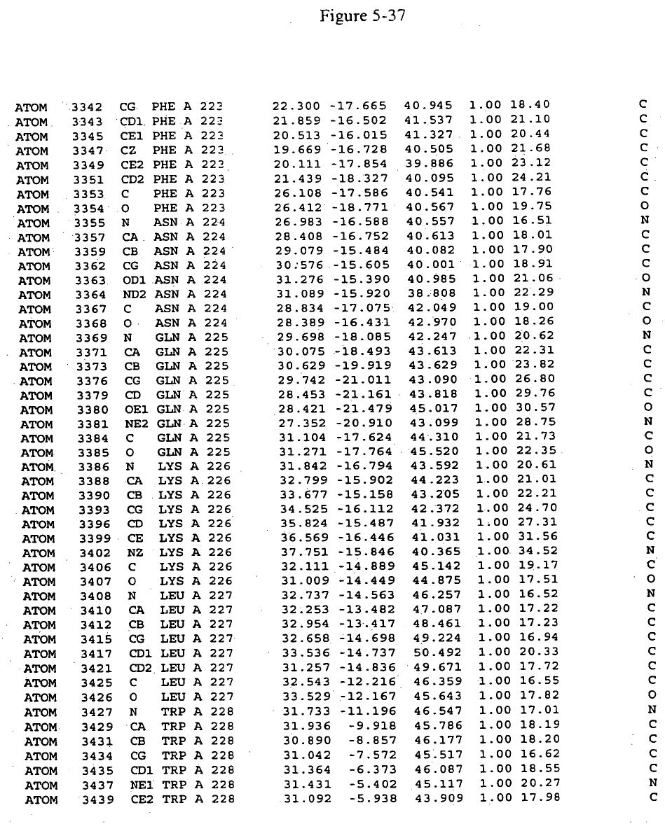

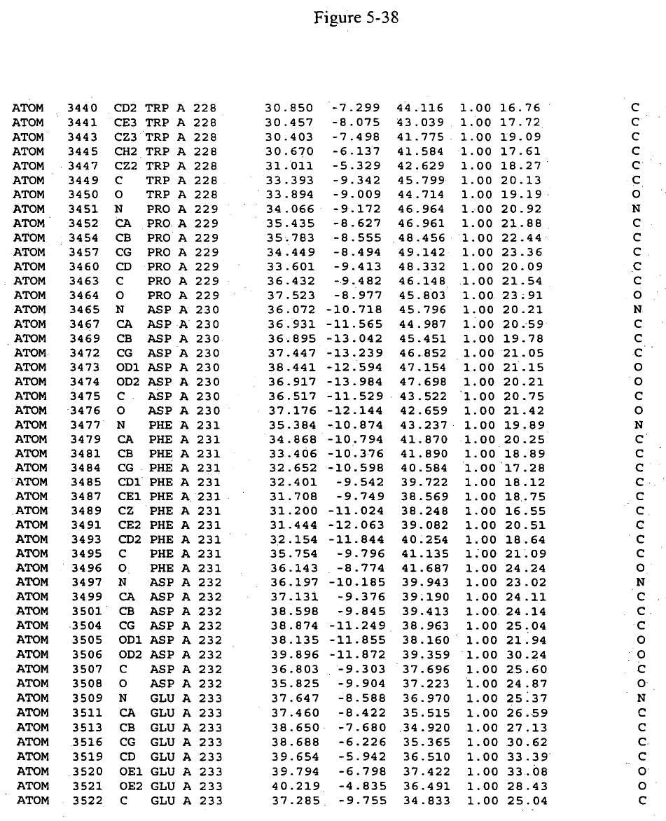

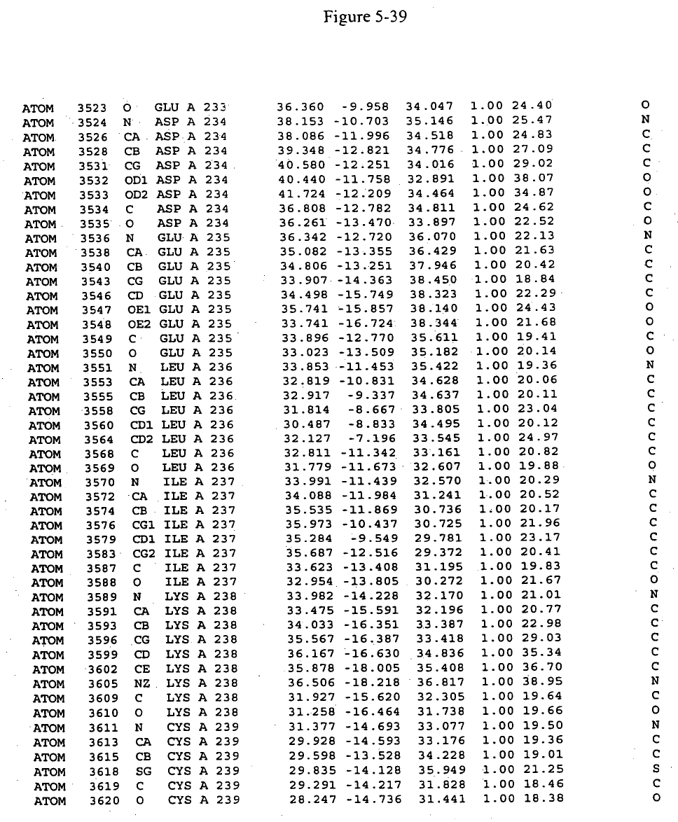

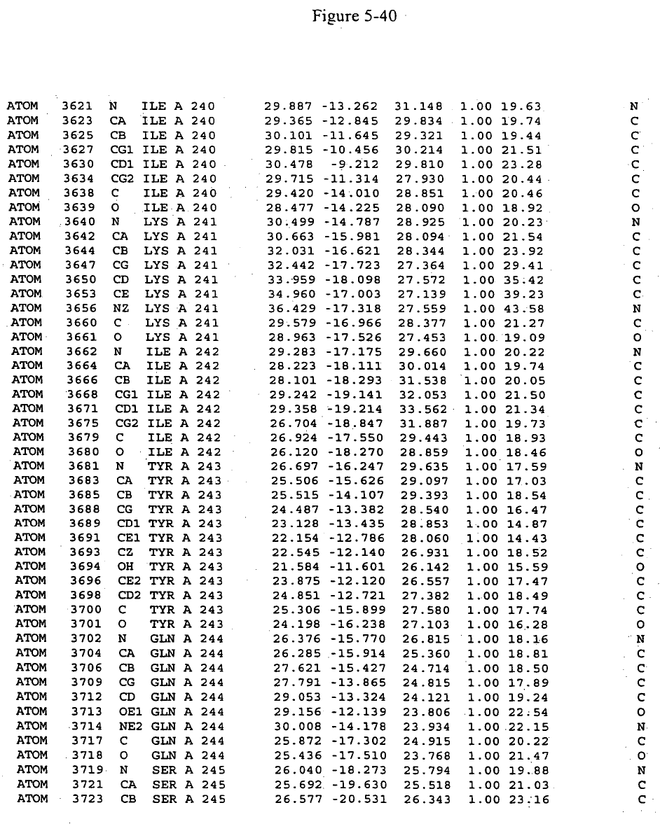

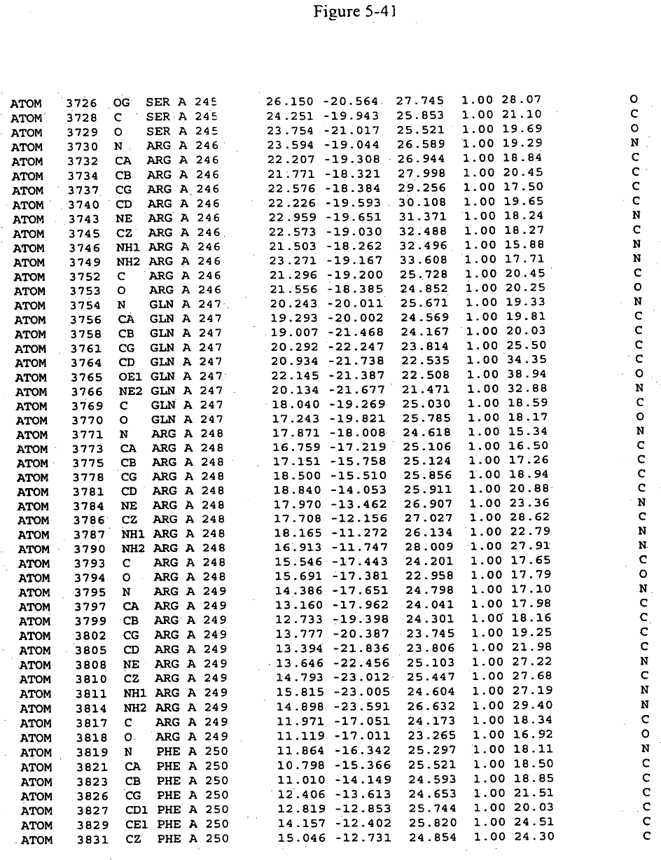

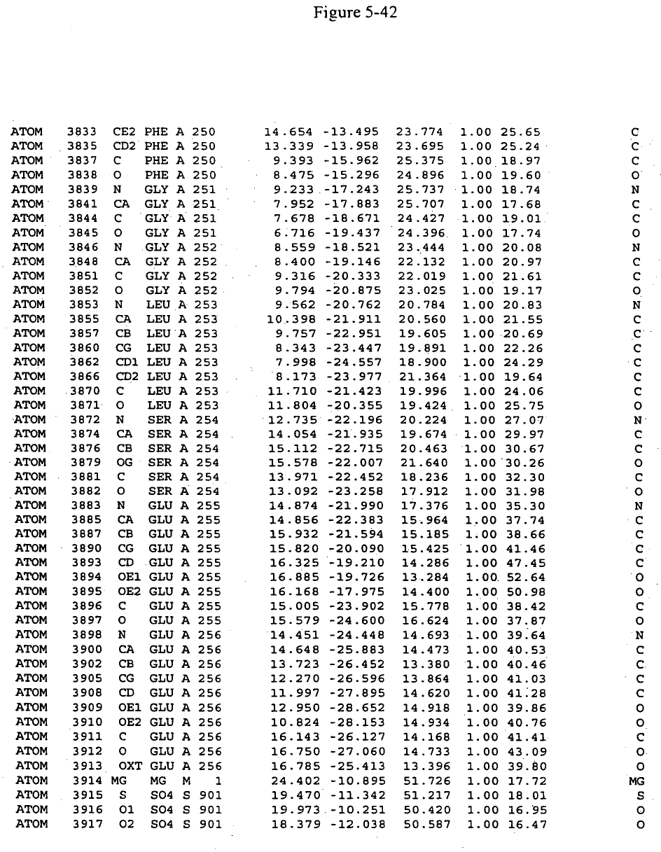

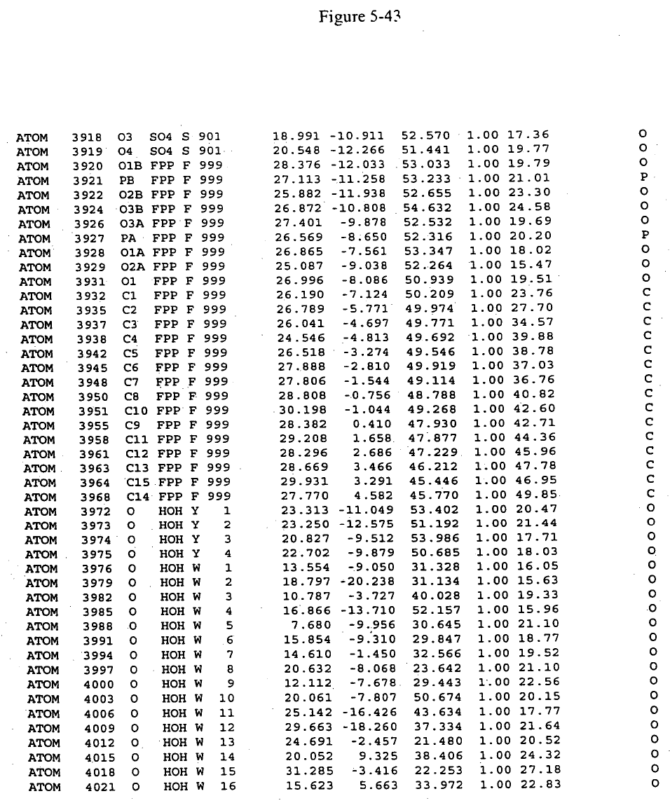

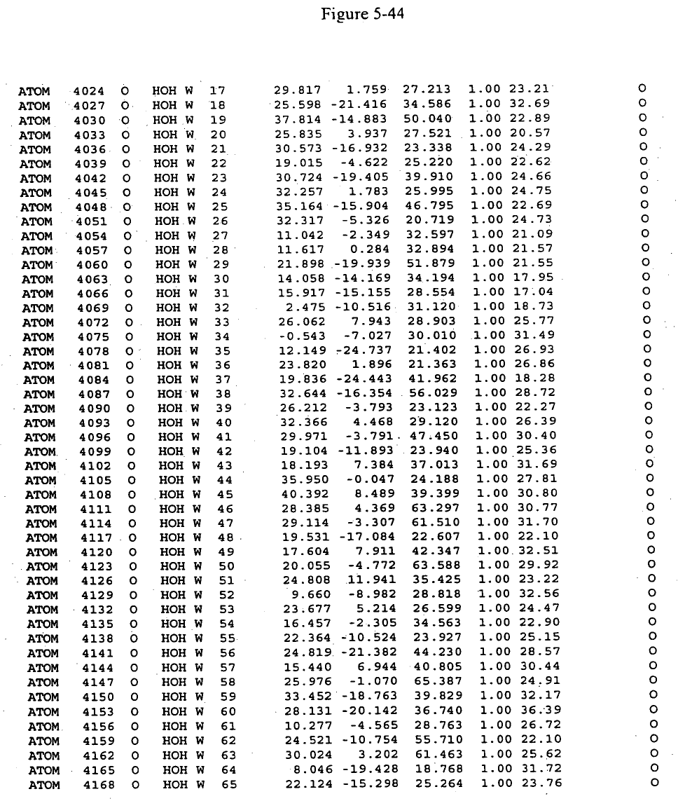

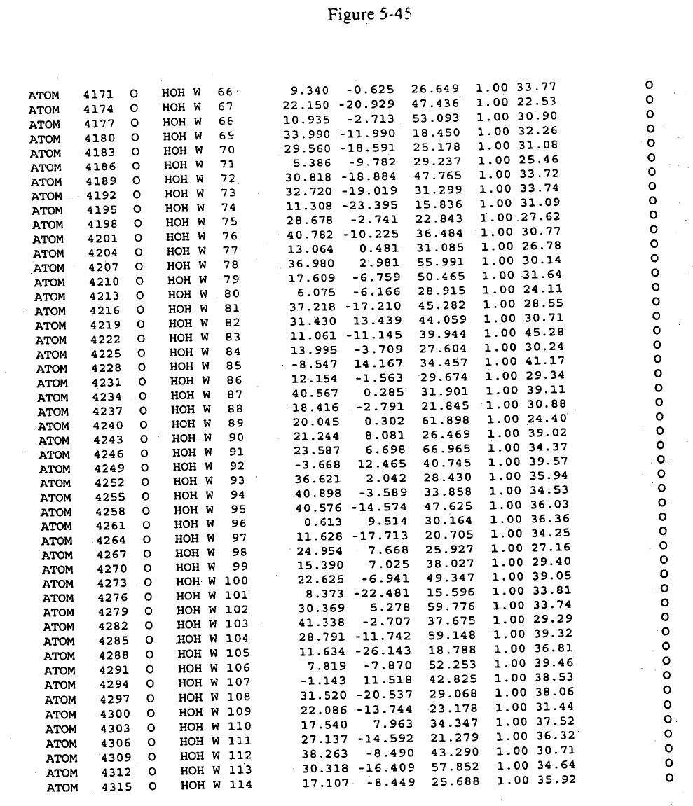

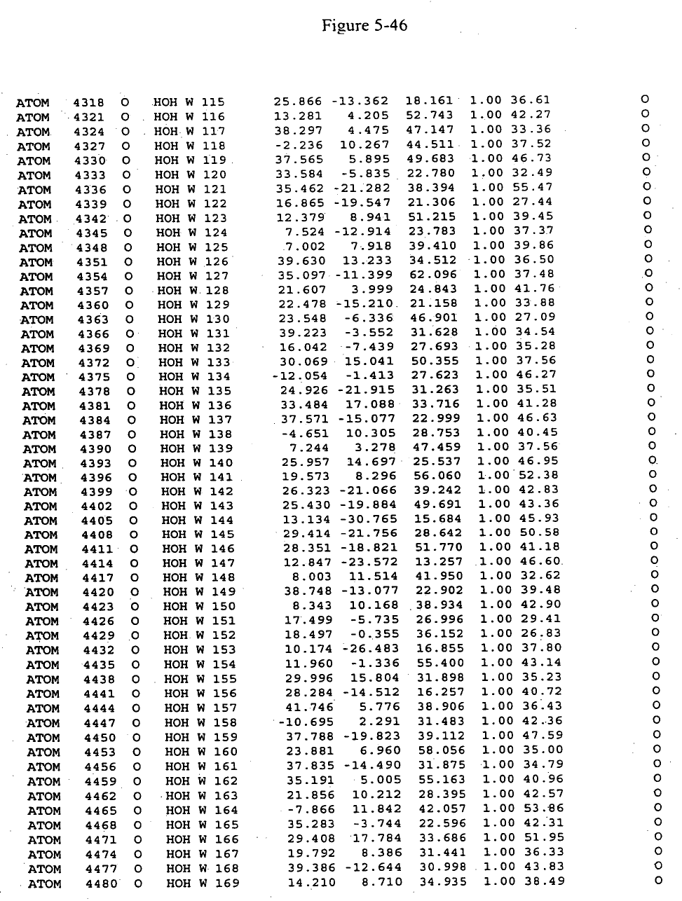

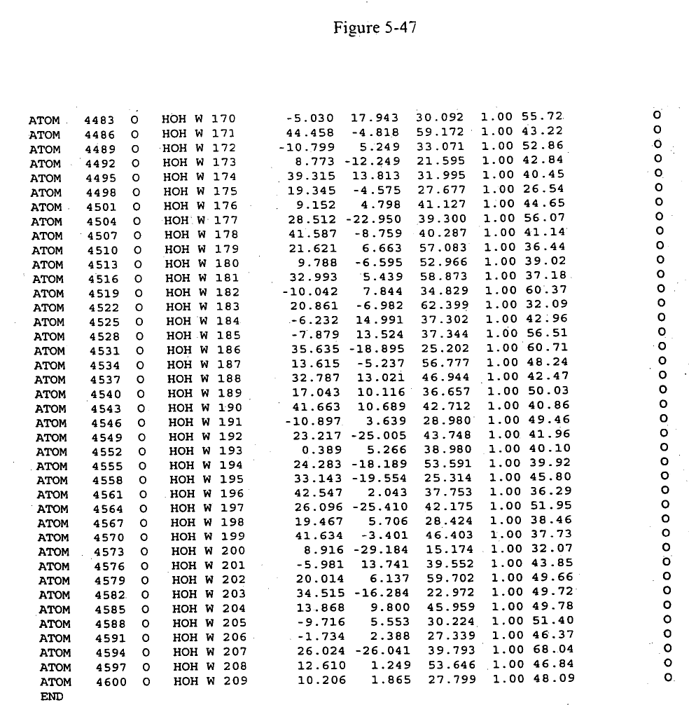

Those of skill in the art understand that a set of structure coordinates determined by X-ray crystallography is not without standard deviation. For the purpose of this invention, any set of structure coordinates for undecaprenyl pyrophosphate synthase or undecaprenyl pyrophosphate synthase homologues or undecaprenyl pyrophosphate synthase mutants that have a root mean square deviation of protein backbone atoms (N, Cₐ, C and O) of less than 0.75 Å when superimposed, using backbone atoms, on the structure coordinates listed in FIG. 5 shall be considered identical.

The term “unit cell” refers to a basic parallelepiped shaped block. The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal.

The term “molecular replacement” refers to a method that involves generating a preliminary model of an undecaprenyl pyrophosphate synthase crystal whose structure coordinates are unknown, by orienting and positioning a molecule whose structure coordinates are known (e.g., undecaprenyl pyrophosphate synthase coordinates from FIG. 5) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subjected to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972). Using the structure coordinates of undecaprenyl pyrophosphate synthase provided by this invention, molecular replacement may be used to determine the structure coordinates of a crystalline mutant or homologue of undecaprenyl pyrophosphate synthase or of a different crystal form of undecaprenyl pyrophosphate synthase.

“Atom type” in, for example, FIG. 5, refers to the element whose coordinates are measured. The first letter in the column in FIG. 5 defines the element.

“X, Y, Z” crystallographically define the atomic position of the element measured.

“B” is a thermal factor that measures movement of the atom around its atomic center.

Atomic coordinates for undecaprenyl pyrophosphate synthase according to FIG. 5 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, and any combination of the above. The atomic coordinates of FIG. 5 correspond to the undecaprenyl pyrophosphate synthase polypeptide chains, and to several molecules bound thereto, including magnesium ion, FPP, sulfate, and a plurality of water molecules.

### MATERIALS AND METHODS

Cloning and Expression

S. aureus UPS was subcloned into the expression vector pET15b (Novagen). Expression was carried out in the E. coli strain BL21IDE3 in M9 minimal media containing seleno-L-methionine. Cells were grown overnight in methionine supplemented M9 seleno-L-methionine media at 37 degrees Celsius. 50 mls of the overnight were inoculated into one liter of seleno-L-methionine labeling media. The cells were grown to an OD₆₀₀ of 0.651 at 37 degrees Celsius and induced with 50 μM IPTG. The cells were allowed to grow for 18 hours at 37 degrees Celsius before harvest. A four-liter growth had a final pellet weight of 16.1 grams.

M9 Seleno-L-methionine labeling media was prepared using the reagents and amounts shown below.

| Reagent | Amount Per Liter |
|---|---|
| 10X M9 salts* | 100 mls |
| Amino Acid Mix** | 100 mls |
| 20% glucose | 20 mls |
| Ampicillin stock (100 mgs/ml in H₂O) | 1 ml |
| 9.8% MgSO₄ (w/v) | 1 ml |
| 10 mM CaCl₂ | 10 mls |
| 2 mg/ml Thiamine | 1 ml |
| 200X Seleno-L-methionine stock*** | 5 mls |

*1X concentration of M9 salts per liter (made up as 10X and autoclaved): 6.0 gm Na₂HPO₄, 3 gm KH₂PO₄, 0.5 gm NaCl and 1.0 gm NH₂Cl.
**Amino acid mix 400 mg/l made up of each of the 19 AA other than methionine (D/L mix) made up in 20 mM NaPO₄ buffer, pH 7.0, filter sterilized.
***200X selono-L-methionine stock made up of 5 mg per ml seleno-L-methionine in 50 mM NaPO₄ buffer, pH 7.0

The components were combined and the final media was filter sterilized through a 0.2 μm filter.

Methionine supplemented M9 seleno-L-methionine media was prepared with identical to reagents as listed above for seleno-L-methionine labeling media except that, instead of 5 mls of 200× seleno-L-methionine stock solution per liter, 2.5 mls of seleno-L-methionine stock and 2.5 mls of L-methionine stock (5 mg per ml L-methionine in 50 mM NaPO₄ buffer, pH 7.0) were added per liter of media.

Purification of Native S. aureus UPS:

Cells from a 1 liter shake flask culture of E. coli expressing the (his)₆-UPS fusion protein were lysed in buffer containing 50 mM TrisCl, 0.3 M NaCl, 4 mM b-ME, pH 8.0, 1 ug/mL pepstatin, 1 ug/mL leupeptin and 1 mM PMSF, using a microfluidizer. Lysate was clarified by centrifugation at 40,000× g. Soluble protein was applied to a 5 mL immobilized metal affinity column, Ni-NTA, which had been equilibrated in buffer A (50 mM TrisCl, 0.3 M NaCl, 4 mM b-ME, 20 mM imidazole, pH 8.0 and 1 ug/mL pepstatin and leupeptin). After washing with buffer A+40 mM imidazole, bound protein was step eluted with buffer B (buffer A+0.25 M imidazole, pH 8.0). Fractions were analyzed by SDS-PAGE on 10% bis-tris gels in MES buffer using the NuPAGE system. Fractions containing his-UPS were pooled for further purification.

The (his)₆ tag was removed by digestion with thrombin at a specific thrombin recognition site. (His)₆-UPS was treated with thrombin at a 1:400 thrombin : (his)₆-UPS ratio. The reaction was stopped by addition of 1 mM PMSF after 2.5 hrs. at room temperature. Chromatography was performed as described above for isolation of (his)₆-UPS. Untagged UPS was isolated in the flowthrough and wash fractions. The remaining (his)₆-UPS and the cleaved (his)₆ peptide were removed in the bound fraction. Fractions were analyzed by SDS-PAGE on 10% bis-tris gels in MES buffer using the NuPAGE system. Fractions containing des-his-UPS were pooled for further purification.

Des-his-UPS was further purified by size exclusion chromatography. The protein was concentrated to 10-12 mg/mL and loaded onto a 125 mL Superdex 200 prep grade column which had been equilibrated in buffer containing 50 mM TrisCl, 0.3 M NaCl, 8 mM DTT, pH 7.5. UPS eluted as a 50 kDa dimer. Fractions were analyzed by SDS-PAGE on 10% bis-tris gels in MES buffer using the NuPAGE system. The final pool was characterized by dynamic light scattering (DLS), and LCMS. DLS revealed that the protein was monodisperse. Mass analysis revealed a small level of truncated N-terminus consistent with the loss of 7 amino acids. This was confirmed by N-terminal sequence analysis. Protein isolated in the manner described above was used for crystallization.

Crystallization of Native S. aureus UPS:

A ternary complex of des-his-UPS: trans-trans-FPP: Mg was made in 50 mM TrisCl, 0.3 M NaCl, 8 mM DTT, pH 7.5. Sparse matrix screening using the hanging drop method was performed. Drops were set at a protein concentration of 5 mg/mL. Plates were incubated at 22° C. Two leads were identified, where protein concentration was 5-8 mg/ml over reservoir containing either 0.1 M Bicine, pH 9, and 2-2.4 M (NH₄)₂SO₄ or 0.1 M NaMES, pH 6.5, 0.2 M (NH₄)₂SO₄, and 30% PEG MME 5000.

Dendritic crystals obtained by spontaneous nucleation grew from drops where the protein: ligand complex was 5 mg/mL over reservoir solution containing 0.1 M NaMES, pH 6.5, 0.2 M (NH₄)₂SO₄, and 30% PEG MME 5000. Seed stocks made from these crystals were used to grow bi-pyramid crystals for x-ray diffraction experiments. Drops of 2 uL complex+2 uL reservoir solution were set with reservoir solutions containing 0.1 M NaMES, pH 6.5, 0.2 M (NH₄)₂SO₄, and 21, 24 and 30% PEG MME 5000. These drops were micro-seeded by serial dilution of the seed stock through 5 identical drops. These drops equilibrated over the appropriate reservoir solution at 22° C. As the seeds diluted out, bi-pyramidal crystals˜250×100 microns grew.

Purification of Selenomethionine Labeled S. aureus UPS:

To solve the structure using the method of MAD-phasing, protein labeled with selenium was expressed. Selenomethionine substituted UPS was purified by the method outlined for native UPS. Mass analysis of the purified untagged product revealed 3 components. The primary species observed was consistent with 7 selenium additions. A minor species was detected consistent with 6 selenium additions. An additional minor species was detected consistent with 6 selenium additions to the N-terminally truncated species (minus 7 amino acids, one of which is a methionine).

Crystallization of Selenomethionine Labeled S. aureus UPS:

The condition used for crystallization of the native UPS was used for labeled UPS Selenomethionine labeled UPS: t-t-FPP: Mg complex was set in drops as described above for the growth of native UPS crystals. Native seeds from dendrites were used to cross seed into drops set using the labeled protein. A second round of micro-seeding was performed using crystals grown from selenomethioine labeled UPS. Using this method, bi-pyramidal crystals were grown for MAD data collection using synchrotron radiation. Data from these crystals were used to solve the structure

The sequence of the construct used in crystallization experiments given in SEQ ID NO:1.

Data Collection

X-ray diffraction data were collected from flash-frozen crystals at 100K. Crystals were briefly soaked in a cryoprotectant solution which consisted of 10% MPD (2,4 methyl pentane diol) added to the crystallization reservoir solution. They were then introduced into a 100° K cold nitrogen stream.

Crystals are tetragonal, belong to the space group P4₁2₁2, with unit cell dimensions of a=58.19, b=58.19, c=159.26 Å.

Three-wavelength diffraction data to a maximum resolution of 1.83 Å were collected at beamline X12C of the National Synchrotron Light Source at Brookhaven National Laboratory, using a Brandeis 2×2 ccd detector. Data were reduced using the HKL suite of programs, and truncated to F's using the program TRUNCATE. Crystal information: Space group P4₁2₁2: Cell dimensions a=b=58.19 Å c=159.26 Å a=b=g=90°. Data statistics are shown in Table 1A below. Values in parentheses are for the highest resolution shell. R_{sym}=S(I-<I>)/S<I>

TABLE 1A

| Crystal | dₘᵢₙ Å | N_{unique} | Redundancy | <I/s> | R_{sym} (%) |
|---|---|---|---|---|---|
| Se-met I1 | 1.83 | 24,214 | 4.4 | 14.5(1.8) | 8.7(43.8) |
| (0. 9789 Å) | | | | | |
| Se-met I2 | 1.83 | 15,892 | 12.3 | 15.5(2.2) | 8.0(38.9) |
| (0.9785 Å) | | | | | |
| Se-met I3 | 1.83 | 16,202 | 12.7 | 16.8(3.0) | 6.8(33.9) |
| (0.9500 Å) | | | | | |

Structure Determination

The synthase structure was solved using the program SOLVE. Se-atom parameter refinement and MAD phasing were carried out using SHARP, and solvent flipped maps were calculated using SOLOMON. Phasing statistics are in shown in Table 1B.

TABLE 1B

| | I1 Centric | I1 Acentric | I2 centric | I2 acentric | I3 centric | I3 Acentric |
|---|---|---|---|---|---|---|
| Phasing power* | 2.7/— | 2.9/2.0 | 3.7/— | 3.6/2.0 | —/— | —/1.8 |
| R_{cuIIis}** | 42.5/— | 51.7/71.9 | 40.7/— | 48.3/71.2 | —/— | —/78.5 |

*Phasing power (isomorphous/anomalous) = <[|Fh(calc)|/E>,
**R_{cullis} (isomorphous/anomalous) = E/<|F_{pH} - Fₚ|>, where E is phase-integrated lack of closure error and F_{PH} and F_{P} are heavy-atom and protein structure factors respectively.

Figure of Merit (FOM) statistics as a function of resolution in shown in Table 1C below.

TABLE 1C

| | Resolution (Å) | | | | | | | | |
|---|---|---|---|---|---|---|---|---|---|
| | | | | | | | | | Over- |
| | 5.09 | 3.63 | 2.97 | 2.58 | 2.31 | 2.11 | 1.95 | 1.83 | all |
| FOM | 0.83 | 0.73 | 0.70 | 0.65 | 0.55 | 0.42 | 0.34 | 0.32 | 0.50 |
| (acen- | | | | | | | | | |
| tric) | | | | | | | | | |
| FOM | 0.71 | 0.58 | 0.55 | 0.52 | 0.51 | 0.39 | 0.32 | 0.28 | 0.48 |
| (centric) | | | | | | | | | |

Automated map-tracing, as implemented in ARP/wARP was used to trace the map, and the program was able to automatically fit 201 out of 259 residues into the 1.83 Å solvent-flipped map. The remaining residues were fit manually, using the program O. All but the first 18 residues were successfully fit into density. Clear electron density was seen for bound FPP and Mg²⁺, as well as for a bound sulfate ion. Bound water molecules were automatically picked using ARP. All model refinement was carried out using the program REFMAC as implemented in the CCP4 suite of programs.

The final refined model consists of residues 19-256 of undecaprenyl pyrophosphate synthase, FPP, Mg²⁺, sulfate, and 222 bound water molecules. This model has an overall R factor of 0.19, with a free R-factor of 0.24, for all data to 1.83 Å. Refinement statistics are in Table 1D.

TABLE 1D

| Resolution | 50.0-1.83 Å |
|---|---|
| Reflections | 22,639 (all data) |
| R_{work} | 19.35% |
| R_{free} | 23.44% |
| RMSD in bond lengths | 0.016 Å |
| RMSD in bond angles | 1.74° |
| Total atoms (non-hydrogen) | 2204 |
| Ramachandran Plot Most favoured | 87.9 |
| Ramachandran Plot Allowed | 12.1 |

Final refined coordinates of S. Aureus UPS are shown in FIG. 5.

The Structure of Staphylococcus Undecaprenyl Pyrophosphate Synthase

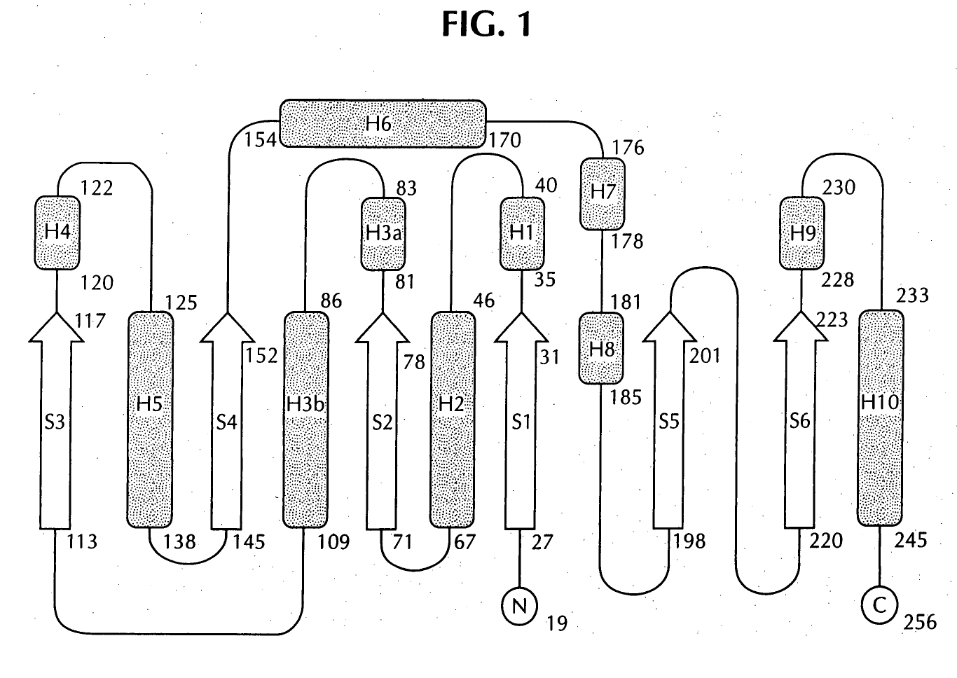

Turning to FIG. 1, the overall topology of the synthase is shown schematically to consist of a six-stranded parallel β-sheet, surrounded by six α-helices and four 3₁₀ helices.

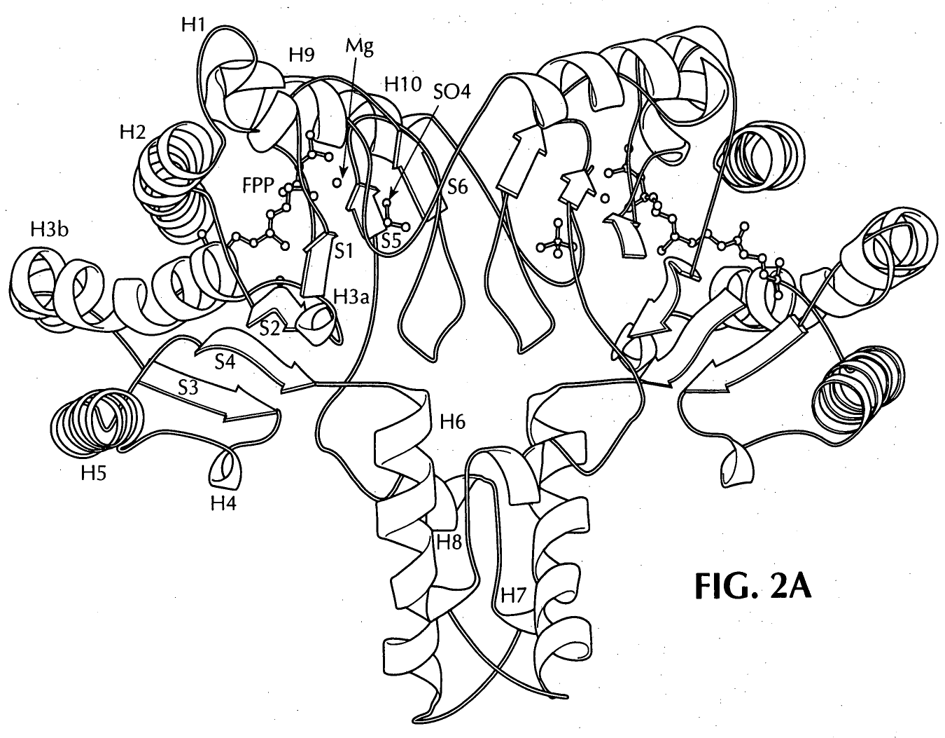

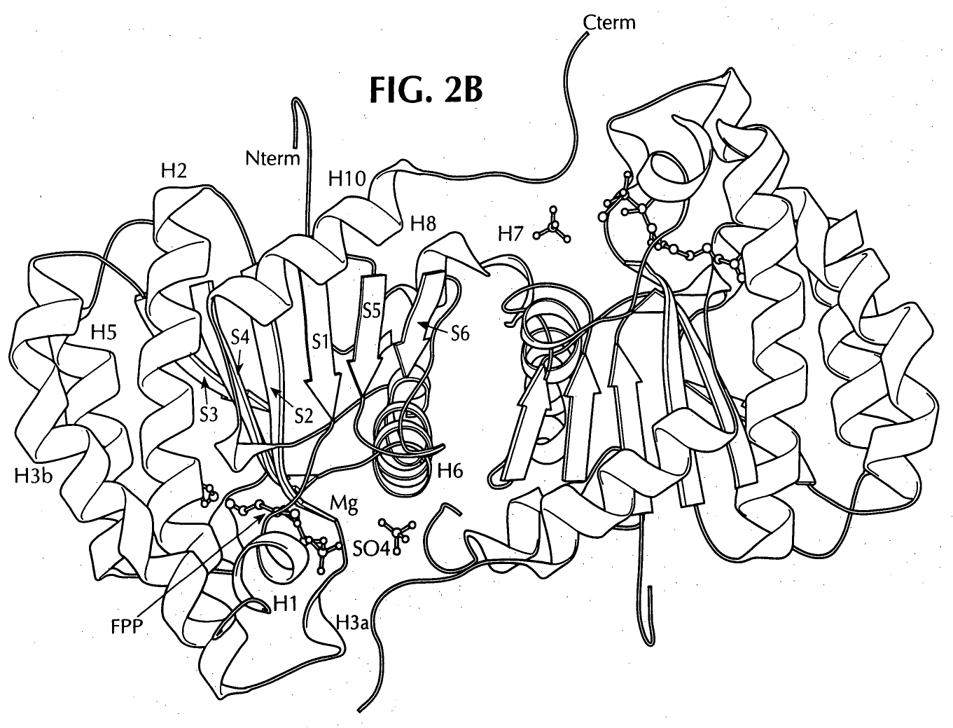

Turning to FIG. 2a and 2b, analysis of the crystal packing reveals that undecaprenyl pyrophosphate synthase exists as a dimer, with two identical subunits related by a 2-fold axis of symmetry. FIGS. 2a and 2b show ribbon drawings of the undecaprenyl pyrophosphate synthase dimer. As depicted in the figures, the two subunits are intimately associated. The ligand binding sites are at the top of the FIG. 2a, as indicated.

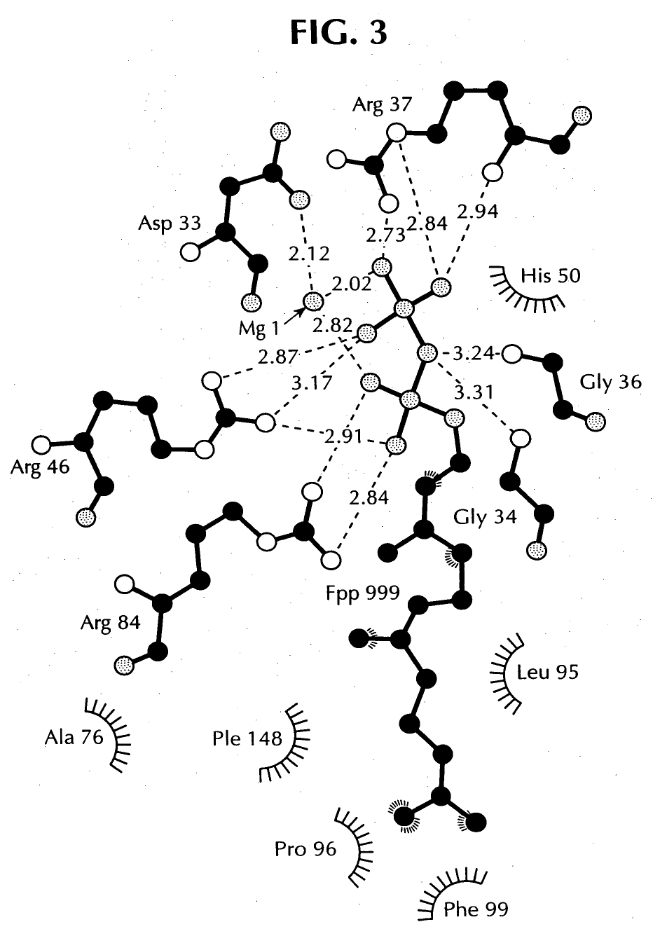

Turning to FIG. 3, the crystal structure reveals details of the binding sites of the substrate FPP and the cofactor Mg²⁺. FIG. 3 shows the detailed interactions of these molecules with the protein, including interatomic distances.

The residues from Ser⁷⁸ to Val⁸⁹ form a coil (specifically a coil-helix-coil, also termed a loop), which is fully ordered in the disclosure of the instant invention, and forms part of the entrance to the catalytic cleft. Arg⁸⁴ makes two hydrogen bonds with a phosphate group of FPP (see FIG. 3). The loop has clearly defined atomic coordinates in the structure according to the instant invention. The loops corresponding to this coil are disordered, that is, not defined, in the structures of undecaprenyl pyrophosphate synthase from M. luteus as determined by Fujihashi et al. (2001) and from E. coli as determined by Ko et al. (2001). Neither Fujihashi nor Ko included substrate or inhibitor in their crystals. This difference suggests that the loop is flexible in the absence of substrate, or other suitable ligand, and the loop will occasionally be referred to as “flexible.”

Moreover, as shown in FIG. 3, the His⁵⁰, Leu⁹⁵, Phe⁹⁹, Pro⁹⁶, Phe¹⁴⁸ and Ala⁷⁶, have hydrophobic interactions with the isoprenoid tail of farnesyl pyrophosphate.

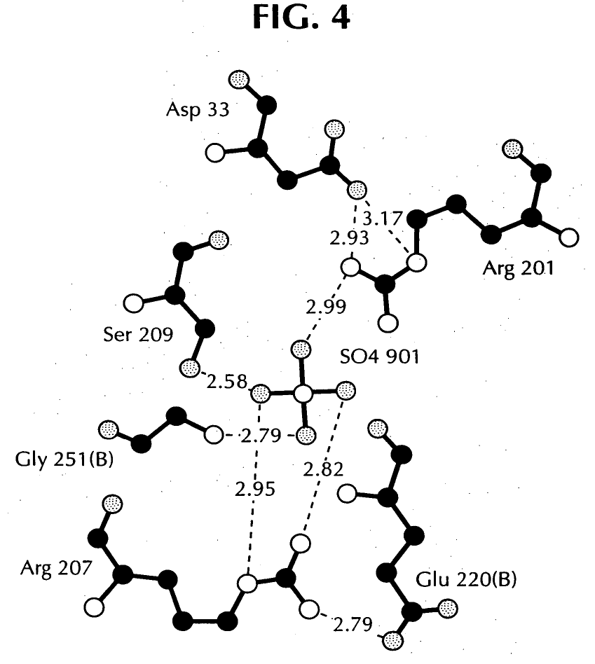

Turning to FIG. 4, a sulfate ion is bound to a second ligand binding site close to the FPP binding pocket. The second ligand binding site is considered to define part of the binding pocket for the second substrate, IPP.

Thus, the active site is defined as consisting of at least one of the following residues: Asp³³, Gly³⁴, Gly³⁶, Arg³⁷, Arg⁴⁶, Ala⁷⁶, Arg⁸⁴, Leu⁹⁵, Pro⁹⁶, Phe¹⁴⁸, Arg²⁰¹, Arg²⁰⁷, and Ser²⁰⁷, from the one chain, and Glu²²⁰ and Gly²⁵¹ from the other chain, denoted chain B. In mutants or homologs of S. aureus undecaprenyl pyrophosphate synthase the numbering of amino acid residues can be normalized to the S. aureus reference sequence.

The S. aureus undecaprenyl pyrophosphate synthase has several structural features indicated in Table 1E below.

TABLE 1E

| | |
|---|---|
| His²⁷-Ile³¹ | S1 |
| Asn³⁵-Lys⁴⁰ | H1 |
| Arg⁴⁶-Ile⁶⁷ | H2 |
| Tyr⁷¹-Ser⁷⁸ | S2 |
| Asn⁸¹-Ser⁸³ | H3a |
| Glu⁸⁶-Glu¹⁰⁹ | H3b |
| Lys¹¹³-Ile¹¹⁷ | S3 |
| Thr¹²⁰-Lys¹²² | H4 |
| Lys¹²⁵-Thr¹³⁸ | H5 |
| Lys¹⁴⁵-Tyr¹⁵² | S4 |
| Gly¹⁵⁴-His¹⁷⁰ | H6 |
| Ser¹⁷⁶-Ile¹⁷⁸ | H7 |
| Glu¹⁸¹-Asn¹⁸⁵ | H8 |
| Leu¹⁹⁸-Arg²⁰¹ | S5 |
| Glu²²⁰-Phe²²³ | S6 |
| Trp²²⁸-Asp²³⁰ | H9 |
| Glu²³³-Ser²⁴⁵ | H10 |

Undecaprenyl Pyrophosphate Synthase Secondary Structure Assignments

In the Table 1E, beta-strands are labeled S1-S6 and helices are labeled H1-H10. The helices H3a, H4, H7 and H9 are 3₁₀ helices; the others are alpha helices. Secondary structures have been calculated according to the method of Kabsch and Sander, as implemented in the program Procheck. Other algorithms used to calculate secondary structure can produce slightly different assignments.

The S. aureus undecaprenyl pyrophosphate synthase has several notable structural features, including the following. The amino acid residues from position 27-31 are part of a beta sheet strand termed S1. H1 is an alpha helix immediately adjacent to a catalytic aspartic acid in position 33 and also has several FPP binding residues including Gly³⁴, Asn³⁵, Gly35 and Arg³⁷. The amino acid residues from position 46 to 67 form an alpha helix termed H2. The amino acid residues from position 71 to 78 are part of a beta sheet strand termed S2. The amino acid residues from position 78 to 89, described above as capable of being a flexible loop, are clearly visible in the structure provided and include Arg⁸⁴ that makes two hydrogen bonds with a phosphate group of FPP. This sequence also includes a 3₁₀ helix and part of an alpha helix when the substrate is present. Thus, the amino acid residues from Asn⁸¹ to Ser⁸³ from a 3₁₀ helix termed H3a.

Moreover, the synthase has other notable features. The amino acids from Glu⁸⁶ to Glu¹⁰⁹ form an alpha helix termed H3b. Of interest, the H3b helix includes a proline that may allow flexibility in the structure. The amino acid residues from Lys¹¹³ to Ile¹¹⁷ form a part of a beta sheet termed S3. The amino acid residues from Thr¹²⁰ to Lys¹²² form a 3₁₀ helix termed H4. The amino acids from Lys¹²⁵ to Thr¹³⁸ form an alpha helix termed H5. The amino acids from Lys¹⁴⁵ to Tyr¹⁵² form part of a beta sheet termed S4. The amino acid residues from Gly154 to His170 form an alpha helix termed H6. The amino acid residues from Ser¹⁷⁶ to Ile¹⁷⁸ form a 3₁₀ helix termed H7. The amino acid residues from Glu¹⁸¹ to Asn¹⁸⁵ form an alpha helix termed H8. The amino acid residues from Leu¹⁹⁸ to Ang²⁰¹ form a strand of beta sheet termed S5. The amino acid residues from Glu²²⁰ to Phe²²³ form a strand of beta sheet termed S6. The amino acid residues from Trp²²⁸ to Asp²³⁰ form a 3₁₀ helix termed H9. The amino acid residues from Glu²³³ to Ser²⁴⁵ form an alpha helix termed H10.

The crystal structure clearly provides a description of the interaction of the amino acid residues of undecaprenyl pyrophosphate synthase with farnesyl pyrophosphate and the magnesium ion cofactor. The Asp³³ has a carboxylic acid functional group in the beta position, the oxygen atom of which interacts with the magnesium ion at a distance of about 2.12 Å. This metal coordination serves to lock the magnesium ion into a position to interact with two oxygen atoms of the pyrophosphate group of farnesyl pyrophosphate at intermolecular distances of about 2.02 Å.

The synthase interaction with FPP is also mediated by other amino acid residues. Arg³⁷ has nitrogen atoms that interact with the oxygen atoms of the phosphates in farnesyl pyrophosphate, with nitrogen to oxygen hydrogen bond interactions having distances of about 2.38 Å, about 2.84 Å and about 2.84 Å. The Gly³⁶ has an alpha amino group that forms a hydrogen bond with the bridge oxygen of farnesyl pyrophosphate having a distance of about 3.24 Å between the nitrogen and oxygen groups. The Gly³⁴ has an alpha amino group that also interacts with the same oxygen atom as shown in FIG. 3 with a nitrogen to oxygen distance of about 3.31 Å. The Arg⁴⁶ has two nitrogen atoms in the guanidino functional group that form hydrogen bonds with an oxygen of the terminal phosphate group in farnesyl pyrophosphate and are characterized by nitrogen to oxygen interatomic distances of about 2.87 and about 3.17 Å. The Arg⁸⁴ has a guanidino group having two oxygens that interact with two oxygen atoms of the phosphate group of farnesyl pyrophosphate forming hydrogen bonds with inter-atomic distances of about 2.84 and about 2.77.

Deficiencies in Other Studies

Comparison of the results of the present invention with deductions by others illustrates the limits of those studies. In regard to the flexible loop, Ko, et al. speculates that the loop is “responsible mainly for IPP binding,” and that Trp-75, corresponding to Trp⁸² in S. aureus undecaprenyl pyrophosphate synthase, “is involved in FPP binding.” The data of the instant invention indicate otherwise. Rather, Arg⁸⁴ forms a pair of hydrogen bonds to FPP. Ala⁷⁶ and Leu⁹⁵, which are adjacent to the flexible loop form hydrophobic interactions with the isoprenoid tail of FPP, but Trp⁸² does not. Ko et al. also states that the glutamate that corresponds to Glu⁸⁸ in S. aureus undecaprenyl pyrophosphate synthase, is critical in substrate binding and/or catalysis. As undecaprenyl pyrophosphate synthase has two substrates, FPP and IPP, Ko et al.'s statement is ambiguous. The data of the instant invention clearly indicate, however, that Glu⁸⁸ makes hydrogen bonds to Arg84 and Arg46, which in turn stabilize the phosphate group of FPP. Thus Glu88 can be said to be indirectly involved in substrate binding.

Fujihashi et al. states that their results indicate that Trp⁸² (in the S. aureus nomenclature) is the residue binding FPP. In contrast the instant invention shows no interaction of Trp⁸⁴ with FPP.

Fujihashi et al. speculates that Arg²⁰¹ and Arg²⁰⁷ (in the S. aureus nomenclature) could interact with the IPP head group, and that, except for these arginines, no conserved residues are found that could bind the pyrophosphate part of IPP. In contrast, the measurements of the instant invention clearly show a hydrogen bond between the hydroxyl group of a high conserved Ser²⁰⁹ and the IPP-mimetic sulfate. Moreover, the instant invention has identified a highly conserved Gly²⁵¹, the nitrogen of which coordinates with the sulfate ligand.

One aspect of the invention relates to a composition comprising Staphylococcus undecaprenyl pyrophosphate synthase in crystalline form and a ligand. In general, the ligand can be a substrate, inhibitor, or co-factor. More specifically, the ligand can be selected from the group consisting of magnesium ion, farnesyl pyrophosphate, isopentyl pyrophosphate, sulfate ion, and any inhibitor that binds to a substrate binding site. The inhibitor can be any inhibitor of the synthase, including a low affinity or high affinity inhibitor. In one aspect the crystal comprises ligands, or parts thereof, having atomic coordinates according to FIG. 5, or portions thereof.

In another aspect the crystalline undecaprenyl pyrophosphate synthase comprises amino acid residues having atomic coordinates according to FIG. 5, or a substantial portion thereof. The synthase is a dimer of identical polypeptide chains. In another aspect, the undecaprenyl pyrophosphate synthase comprises an amino acid sequence corresponding to residues 1-275 of SEQ ID NO:1. SEQ ID NO:1 is the amino acid sequence of S. aureus undecaprenyl pyrophosphate synthase, using the three letter amino acid notation. The amino acid denoted number 12 is the first amino acid of the wild-type sequence. The amino acids denoted 1-11 denote amino acids used in the construct.

The invention also relates to first and second ligand binding sites of undecaprenyl pyrophosphate synthase. The first and second ligand binding sites are defined by amino acid residues that interact with the polar or ionic head group of the ligands and, optionally, with other amino acid residues that interact with a hydrophobic tail of the ligand. Alternatively, the amino acid residues of the binding sites can interact indirectly with the substrate, for example, by binding to a cofactor which in turn binds to a substrate, or by binding to another amino acid residue which in turn binds to a substrate.

The first ligand binding site can be defined as comprising at least one amino acid residue selected from the group consisting of Asp³³, Gly³⁴, Gly³⁶, Arg³⁷, Arg⁴⁶, Ala⁷⁶, Arg⁸⁴, Leu⁹⁵, Pro⁹⁶, and Phe¹⁴⁸. In a preferred embodiment, the first ligand binding site comprises at least three of these amino acid residues. In a yet more preferred embodiment, the first ligand binding site comprises at least six of these amino acid residues. In a most preferred embodiment, the first ligand binding site comprises all ten amino acid residues.

The first ligand binding site can alternatively comprise at least about 80% of the amino acid residues selected from the group consisting of Asp³³, Gly³⁴, Gly³⁶, Arg³⁷, Arg⁴⁶, Ala⁷⁶, Arg⁸⁴, Leu⁹⁵, Pro⁹⁶, and Phe^{148.} In a preferred embodiment, the first ligand binding site comprises at least about 90% of the amino acid residues selected from the group consisting of Asp³³, Gly³⁴, Gly³⁶, Arg³⁷, Arg⁴⁶, Ala⁷⁶, Arg⁸⁴, Leu⁹⁵, Pro⁹⁶, and Phe¹⁴⁸.

The second ligand binding site can be defined as comprising at least one amino acid residue selected from the group consisting of Asp³³, Arg²⁰¹, Arg²⁰⁷, and Ser²⁰⁹ from one chain (A) of the dimer, and Glu²²⁰ and Gly²⁵¹ from the other chain (B) of the dimer. In a preferred embodiment, the second binding site comprises at least three of these amino acid residues. In a more preferred embodiment, the second binding site comprises all of these amino acid residues.

The second ligand binding site can alternatively comprise at least about 80% of the amino acid residues selected from the group consisting of Asp³³, Arg²⁰¹, Arg²⁰⁷, Ser²⁰⁹, Glu²²⁰(B), and Gly²⁵¹(B). In a preferred embodiment, the second ligand binding site comprises at least about 90% of the amino acid residues selected from the group consisting of Asp³³, Arg²⁰¹, Arg²⁰⁷, Ser²⁰⁹, Glu²²⁰(B), and Gly²⁵¹(B).

Another aspect of the invention relates to a method of designing or identifying a potential ligand for an undecaprenyl pyrophosphate synthase, the method comprising using a three-dimensional structure including atomic coordinates of amino acid residues 33, 34, 36, 46, 76, 84, 95, 96, and 148, according to FIG. 5. In a preferred embodiment, the coordinates are those of S. aureus undecaprenyl pyrophosphate synthase, or a substantial portion thereof. The method can include obtaining the potential ligand which can include synthesizing the ligand in whole or in part, borrowing the ligand, and purchasing the ligand.

In one aspect the invention is directed to a computational model of a composition comprising an undecaprenyl pyrophosphate synthase having atomic coordinates of S. aureus undecaprenyl pyrophosphate synthase, or a portion thereof, and a computer program running on a computer addressing the atomic coordinates. The atomic coordinates can be those of FIG. 5, or a substantial portion thereof.

In another aspect, the invention is directed to a method of designing or identifying a ligand or a potential inhibitor of a second undecaprenyl pyrophosphate synthase comprising: (a) using a three-dimensional structure of a first undecaprenyl pyrophosphate synthase, as defined by atomic coordinates according to FIG. 5, or a substantial portion thereof; (b) identifying at least one first amino acid residue having a first peptide backbone and the amino acid residue(s) defining, in part, in at least one ligand binding site; (c) employing protein alignment means to identify in the second undecaprenyl pyrophosphate synthase at least one second amino acid residue having a second peptide backbone that is capable of substantially aligning with the first backbone; (d) employing the three-dimensional structure to design or select the potential ligand for the second undecaprenyl pyrophosphate synthase; (e) synthesizing the potential ligand; and (f) contacting the potential ligand with the second undecaprenyl pyrophosphate synthase to determine binding to the second undecaprenyl pyrophosphate synthase; wherein the second amino acid residue differs from the first amino acid residue.

In yet another aspect, the invention is directed to a computational model of an active site of an isolated undecaprenyl pyrophosphate synthase comprising a magnesium ion cofactor and a polypeptide comprising a first arginine residue having a guanidino group having nitrogen atoms, and an aspartic acid residue comprising oxygen atoms forming an acid functional group, wherein the oxygen atoms coordinate with the cofactor and at least one nitrogen atom of the guanidino group of the first arginine residue; and a second arginine residue in a polypeptide loop comprising the sequence Glu Asn Trp Xaa Arg Pro (SEQ ID NO:2). The Arg has at least one nitrogen atom capable of coordinating an atom of a ligand. Xaa is any amino acid residue, including hydrophilic, hydrophobic, and ionic amino acid residues. The cofactor, aspartic acid residue, first arginine residue, and second arginine residue form at least a part of an active site of an undecaprenyl pyrophosphate synthase. In one embodiment Xaa is Ser.

Moreover, the active site can further comprise a third arginine in an alpha-helix comprising the sequence Asp Gly Asn Xaa Arg (SEQ ID NO:3), the Arg having at least two nitrogen atoms capable of coordinating an atom of a ligand. Xaa in SEQ ID NO:3 is any amino acid residue, including hydrophilic, hydrophobic, and ionic amino acid residues. In one embodiment, Xaa is Gly.

The invention is also directed to a computational model of an active site comprising a representation of the active site of undecaprenyl pyrophosphate synthase by a computer program capable of running on a computer.

In one aspect, the invention is directed to a computational model of a composition comprising an undecaprenyl pyrophosphate synthase having at least twelve of the atomic coordinates of S. aureus undecaprenyl pyrophosphate synthase and a computer program running on a computer addressing the atomic coordinates. Preferably, the model comprises at least twenty-four, more preferably at least 36 atomic coordinates, and most preferably at least 48 atomic coordinates.

The computational model can further comprise an amino acid residue sequence Asp Gly Asn Gly Arg Trp (SEQ ID NO:4), the Arg having at least one nitrogen atom, and a ligand comprising at least one oxygen atom, wherein the at least one nitrogen atom abuts the oxygen atom by about 2.4 Å.

In another embodiment, the computational model can further comprise an amino acid residue sequence Asp Gly Asn Gly Arg Trp (SEQ ID NO:4), each Gly having a nitrogen atom, and a ligand comprising at least one oxygen atom, wherein the nitrogen atom abuts the oxygen by about 3.3 Å.

In yet another embodiment, the computational model can alternatively further comprise an amino acid residue sequence Glu Asn Trp Ser Arg Pro (SEQ ID NO:5), the Arg having at least one nitrogen atom, and a ligand comprising at least one oxygen atom, wherein the nitrogen atom abuts the oxygen atom by about 2.9 Å.

In still another embodiment, the computational model can alternatively further comprise an amino acid residue sequence Pro Arg Ile Lys Gly His (SEQ ID NO:6), the Arg having at least one nitrogen atom, and a ligand comprising at least one oxygen atom, wherein the nitrogen atom abuts the oxygen atom by about 3 Å.

Also, the computational model can alternatively further comprise an amino acid residue sequence Arg Tyr Ser Asn Phe Leu (SEQ ID NO:7), the Ser having one nitrogen atom, and a ligand having at least one oxygen atom wherein the nitrogen atom abuts the oxygen atom by about 2.6 angstroms.

Design of Undecaprenyl Pyrophosphate Synthase Inhibitors

One of skill in the art of molecular modeling can use any method to screen chemical moieties for the ability to associate with undecaprenyl pyrophosphate synthase or with the Mg²⁺, FPP, or IPP binding sites that comprise part of the undecaprenyl pyrophosphate synthase active site. Visual inspection of a model of the ligand binding sites based on the undecaprenyl pyrophosphate synthase coordinates in FIG. 5 can lead to candidate chemical entities. Selected chemical moieties can then be positioned in orientations within one of the ligand binding sites of undecaprenyl pyrophosphate synthase. Positioning can be accomplished using software such as Quanta and Sybyl and is useful for changing the positions of chemical entities. Then standard molecular mechanics forcefields, such as CHARMM and AMBER can be used to minimize the energy and molecular kinetics of binding.

Other computer programs useful in selecting chemical moieties include:

1. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

2. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

3. AUTODOCK (Goodsell, D. S. and A. J. Olsen, “Automated Docking of Substrates to Proteins by Simulated Annealing”, Proteins: Structure. Function, and Genetics, 8, pp. 195-202 (1990)). AUTODOCK is available from Scripps Research Institute, La Jolla, Calif.

4. MCSS (Miranker, A. and M. Karplus, “Functionality Maps of Binding Sites: A Multiple Copy Simultaneous Search Method.” Proteins: Structure. Function and Genetics, 11, pp. 29-34 (1991)). MCSS is available from Molecular Simulations, Burlington, Mass.

Selected moieties can be assembled into a single compound by initial visual review of the organization of the parts to make a whole in relation to the atomic coordinates of undecaprenyl pyrophosphate synthase. Model building with software such as Quanta or Sybyl can supplement the process.

Other programs useful in building chemical moieties into a ligand or inhibitor include:

1. CAVEAT (Bartlett, P. A. et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.

2. 3D Database systems such as MACCS-3D (MDL Information Systems, San Leandro, Calif.). See also, Martin, Y. C., “3D Database Searching in Drug Design”, J. Med. Chem., 35, pp. 2145-2154 (1992)).

3. HOOK (available from Molecular Simulations, Burlington, Mass.).

An undecaprenyl pyrophosphate synthase inhibitor or ligand can be prepared one moiety at a time, as described. Moreover, inhibitory or other undecaprenyl pyrophosphate synthase binding compounds can be designed “de novo” using either a vacant binding site or with moieties of a known inhibitor. Computer programs that support this approach include:

1. LEGEND (Nishibata, Y. and A. ltai, Tetrahedron, 47, p. 8985 (1991)). LEGEND is available from Molecular Simulations, Burlington, Mass.

2. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.

3. LeapFrog (available from Tripos Associates, St. Louis, Mo.).

Variations on molecular modeling can be useful in this invention and include: Cohen, N. C. et al., “Molecular Modeling Software and Methods for Medicinal Chemistry”, J. Med. Chem., 33, pp. 883-894 (1990); and Navia, M. A. and M. A. Murcko, “The Use of Structural Information in Drug Design”, Current Opinions in Structural Biology, 2, pp. 202-210 (1992).

The efficiency of a model ligand binding to undecaprenyl pyrophosphate synthase can be evaluated and optimized by computation. For example, an effective undecaprenyl pyrophosphate synthase inhibitor can induce a relatively small deformation upon binding, that is, the energy in the bound and free states would be similar. Thus, in one embodiment undecaprenyl pyrophosphate synthase inhibitors should preferably have a deformation energy upon binding of about 8 kcal/mole or less. In the case where undecaprenyl pyrophosphate synthase inhibitors can bind to the synthase in more than one conformation the deformation binding energy is the difference between the average energy of the bound conformations less the energy in free solution. Further enhancement of binding can be achieved by computational repulsive charge interaction between the ligand and the synthase. In a similar manner, dipole-dipole interactions can be reduced. Advantageously, the net dipole-dipole and charge interactions between ligand and undecaprenyl pyrophosphate synthase favor binding.

Computer software useful to evaluate energies of deformation and of electrostatic repulsion and attraction include: Gaussian 92, revision C, M. J. Frisch, Gaussian, Inc., Pittsburgh, Pa. ©1992; AMBER, version 4.0, P. A. Kollman, University of California at San Francisco, ©1994; QUANTA/CHARMM, Molecular Simulations, Inc., Burlington, Mass. ©1994; and Insight II/Discover (Biosysm Technologies Inc., San Diego, Calif. ©1994). These applications can be used on suitable workstations. Other hardware systems and software packages will be known to those skilled in the art.

A model undecaprenyl pyrophosphate synthase-binding compound can then be modified by changing functional groups to improve binding or inhibitory properties. The modified group can be similar to the size, volume and distribution of polar and hydrophobic functional groups as the model compound or it can differ. Modified compounds can be analyzed for fit to undecaprenyl pyrophosphate synthase by the computer modeling methods described above.

One aspect of the invention comprises a method of identifying an inhibitor capable of binding to and inhibiting the enzymatic activity of an undecaprenyl pyrophosphate synthase, comprising: (a) introducing into a suitable computer program information defining the binding site of the undecaprenyl pyrophosphate synthase comprising first atomic coordinates of amino acids capable of binding to a substrate, wherein the program displays the three-dimensional structure thereof; (b) creating a three dimensional model of a test compound in the computer program; (c) displaying and superimposing the model of the test compound on the structure of the active site; (d) assessing whether the test compound model fits spatially into the active site; (e) incorporating the test compound in a biological synthase activity assay; and (f) determining whether the test compound inhibits enzymatic activity in the assay.

The method can further comprise introducing into the computer program second atomic coordinates of water molecules bound to the substrate. Thereby, the free energy of binding of the potential inhibitor can include displacement of bound water.

In one embodiment, the method comprises introducing into the computer program an amino acid residue sequence of the synthase, or portion thereof. In one preferred embodiment, the method comprises introducing into the computer program third atomic coordinates of a first 3₁₀ helix of the synthase, comprising the sequence Asn-Trp-Ser. In another embodiment, the method further comprises introducing into the computer program fourth atomic coordinates of at least one synthase structural element selected from the group consisting of an alpha helix, a second 3₁₀ helix, a strand of beta sheet, and a coil.

In yet another embodiment of the method, the undecaprenyl pyrophosphate synthase structural elements consist essentially of a coil and (a) a first beta sheet strand consisting of His Ile Ala Ile Ile (SEQ ID NO:8), or homolog thereof, and a second coil; (b) a first alpha helix consisting of Asn Gly Arg Trp Ala Lys (SEQ ID NO:9), or homolog thereof, and a third coil; (c) a second alpha helix consisting of Arg Ile Lys Gly His Tyr Glu Gly Met Gln Thr Ile Lys Lys Ile Thr Arg Val Ala Ser Asp Ile (SEQ ID NO:10), or homolog thereof, and a fourth coil; (d) a second beta sheet strand consisting of Tyr Leu Thr Leu Tyr Ala Phe Ser (SEQ ID NO:11), or homolog thereof, and a fifth coil; (e) a first 3₁₀ helix consisting of Asn Trp Ser, and a sixth coil; (f) a third alpha helix consisting of Glu Ser Glu Val Asn Tyr Ile Met Asn Leu Pro Val Asn Phe Leu Lys Thr Phe Leu Pro Glu Leu Ile Glu (SEQ ID NO:12), or homolog thereof, and a seventh coil; (g) a third beta sheet strand consisting of Lys Val Glu Thr Ile (SEQ ID NO:13), or homolog thereof, and an eighth coil; (i) a second 3₁₀ helix consisting of Thr Asp Lys, and a ninth coil; (j) a fourth alpha helix consisting of Lys Ser Thr Ile Glu Ala Ile Asn Asn Ala Lys Glu Lys Thr (SEQ ID NO:14), or homolog thereof, and a tenth coil; (k) a fourth beta sheet strand consisting of Lys Leu Ile Phe Ala Ile Asn Tyr (SEQ ID NO:15), or homolog thereof, and an eleventh coil; (l) a fifth alpha helix consisting of Gly Arg Ala Glu Leu Val His Ser Ile Lys Asn Met Phe Asp Glu Leu His (SEQ ID NO:16), or homolog thereof, and a twelfth coil; (m) a third 3₁₀ helix consisting of Ser Asp Ile, and a thirteenth coil; (n) a sixth alpha helix consisting of Glu Thr Tyr Ile Asn (SEQ ID NO:17), or homolog thereof, and a fourteenth coil; (o) a fifth beta sheet strand consisting of Leu Leu Ile Arg (SEQ ID NO:18), or homolog thereof, and a fifteenth coil; (p) a sixth beta sheet strand consisting of Glu Phe Ile Phe (SEQ ID NO:19), or homolog thereof, and a sixteenth coil; (q) a fourth 3₁₀ helix consisting of Trp Pro Asp, and a seventeenth coil; and/or (r) a seventh alpha helix consisting of Glu Asp Glu Leu Ile Lys Cys Ile Lys Ile Tyr Gln Ser (SEQ ID NO:20), or homolog thereof, and an eighteenth coil.

In still another embodiment of the method, the second coil is connected to the first alpha helix, the third coil is connected to the second alpha helix, the fourth coil is connected to the second beta sheet strand, the fifth coil is connected to the first 3₁₀ helix, the sixth coil is connected to the third alpha helix, the seventh coil is connected to the third beta sheet strand, the eighth coil is connected to the second 3₁₀ helix, the ninth coil is connected to the fourth alpha helix, the tenth coil is connected to the fourth beta sheet strand, the eleventh coil is connected to the fifth alpha helix, the twelfth coil is connected to the third 3₁₀ helix, the thirteenth coil is connected to the sixth alpha helix, the fourteenth coil is connected to the fifth beta sheet strand, the fifteenth coil is connected to the sixth beta sheet strand, the sixteenth coil is connected to the fourth 3₁₀ helix, and/or the seventeenth coil is connected to the seventh alpha helix. In this description, the numerical adjectives, first, second and so forth, do not necessarily indicate a temporal or spatial order, but rather, serve merely to distinguish otherwise similarly named elements from one another.

Knowledge of the three-dimensional structure allows solution, by the method of molecular replacement, of crystal structures of undecaprenyl pyrophosphate synthase bound to inhibitors, and use of the method of difference Fourier analysis to determine the bound conformation of the inhibitors. Knowledge of the bound conformation then allows for the design of inhibitors with better properties.

Knowledge of the three-dimensional structure allows the user to solve, by the method of molecular replacement, the structure of undecaprenyl pyrophosphate synthase from any other organism.

Knowledge of the three-dimensional structure allows the user to solve, by the method of molecular replacement, the structures of undecaprenyl pyrophosphate synthase mutants which may be used as probes of undecaprenyl pyrophosphate synthase activity.

### EXAMPLES

The following non-limiting examples are presented to further illustrate the invention.

### Example 1

### Design of an Inhibitor

The atomic coordinates of the polypeptide chains of S. aureus undecaprenyl pyrophosphate synthase, as identified in FIG. 5, can be used in a computer to construct a three-dimensional model of the active site. A putative competitive inhibitor can be fit into a binding site on the enzyme. One such putative inhibitor is (2Z,6E,10E)4-methyl-geranylgeranyl diphosphate. Ohnuma et al., 1989 Kinetic studies on the prenyl chain elongation by undecaprenyl pyrophosphate synthase with artificial substrate homologues. 271 FEBS Lett 257 71. Modifications in the putative inhibitor can be made to prepare a virtual library of structurally related compounds. A docking program can then be used to evaluate interaction of each compound with the synthase, and to compare and rank the relative binding of the compounds to the synthase.

Compounds that appear to have relatively high affinity for the synthase can be obtained or synthesized and evaluated in a biochemical or biological assay. A suitable biological assay can be a measurement of growth by, for example, changes in turbidity of a bacterial suspension culture.

### Example 2

### Use of an Inhibitor of Undecaprenyl Pyrophosphate Synthase Activity to Identify Novel Ligands

Novel ligands capable of binding to an undecaprenyl pyrophosphate synthase substrate binding site can be identified by using a known inhibitor, for example, (S)-farnesyl thiopyrophosphate, or a substrate, for example, farnesyl pyrophosphate. Useful substrates of the synthase in addition to isoprenyl pyrophosphate (C₅PP) and farnesyl pyrophosphate (C₁₅PP) include C₂₀PP, C₂₅PP, C₃₀PP, C₃₅PP, C₄₀PP, C₄₅PP, and C₅₀PP, where the subscript denotes the number of carbon atoms in the isoprenoid chain. Properties of (S)-farnesyl thiopyrophosphate are described by Chen et al. Chen YH et al. Probing the conformational change of Escherichia coli undecaprenyl pyrophosphate synthase during catalysis using an inhibitor and tryptophan mutants. 277 J.Biol.Chem. 7369 (2002).

The atomic features of the known inhibitors or substrates are introduced into a suitable computer program that has information defining the substrate binding site. Typically, the information includes atomic coordinates of those amino acids that can bind to a known synthase substrate, such as are identified in FIG. 5. The computer program can then display the three-dimensional structure of the binding site. Then a three-dimensional model of a test compound can be created in the computer program.

A docking program can be used to dock the model of the test compound to the structure of the binding site. That is, the program fits the test molecule into the binding site, allowing for rotation of the bonds of the molecule to test the several conformation of the test molecule, and evaluates the quality of fit. Similarly, a three dimensional model of the substrate or of an inhibitor of the synthase can be created and docking information obtained. Then the docking parameters of the test compound can be compared to those of the substrate or of the known inhibitor. The docking program can then provide an output which can rank order the association parameters of each test or comparison molecule to the synthase.

In consequence, candidate compounds most likely to have high affinity for the binding site can be readily identified. Synthesis of the most potent test molecules, or otherwise obtaining them, can provide physical molecules for biochemical or biological analysis.

The method can optionally include introducing the atomic coordinates of those water molecules bound to the substrate, such that the coordinates are available to the computer program. Optionally, one skilled in the art can introduce into the computer program the atomic coordinates of at least one synthase structural element. Exemplary structural elements are an alpha helix, a 3₁₀ helix, a strand of beta sheet, and a coil. The 3₁₀ helix can have the sequence Asn Trp Ser, a part of a polypeptide loop that can engage the substrate.

The method can optionally also include incorporating the test compound in a biochemical synthase activity assay for a synthase; and then determining whether the test compound inhibits synthase activity in the assay. Suitable inhibitors can be further assessed in cell permeability studies, viability studies, and bacteremia studies, for example by biological assays.

### Example 3

### Undecaprenyl Pyrophosphate Synthase Assay

Undecaprenyl pyrophosphate synthase activity can be determined by standard methods. Measurement of synthase activity in vitro in the absence and presence of putative inhibitors can yield information on direct effects on the synthase. By comparison, measurements using viable bacteria in the absence and presence of putative inhibitors can yield information, when compared with in vitro analyses, of cell permeation. One skilled in the art will recognize that undecaprenyl pyrophosphate synthase substrates and close analogs thereof will be substantially cell impermeant under normal conditions.

One suitable method of in vitro analysis follows. [¹⁴C]-IPP (55 mCi/mmol) is incubated for up to 20 min at 25° C. in the presence of IPP (2-400 μM), FPP (0.2-10 μM), synthase (0.01-0.1 μM) in a suitable buffered solution. A suitable buffered solution is 0.1% Triton X-100 in 50 mM KCl, 0.5 mM MgCl₂,100 mM KOH-HEPES, pH 7.5. To measure a rate, aliquots of the reaction mixture are removed at timed intervals and mixed with a solution of 10 mM EDTA to stop the reaction. The reaction products are extracted with 1-butanol, the phases separated, and the radioactive materials measured by scintillation counting. The butanol phase, which contains the undecaprenyl pyrophosphate, can be evaporated, and the pyrophosphate groups hydrolyzed in a solution of 20% propanol containing 4.4 units/ml acid phosphatase, 0.1% Triton X-100, and 50 mM sodium acetate, pH 4.7. The resultant polyprenols are extracted with 1-hexane and spotted on a reversed-phase TLC plate and developed using acetone/water (19:1) as the mobile phase. The TLC plates are then analyzed by autoradiography.

One skilled in the art can measure synthase activity in vitro using the assay described above in the absence and presence of putative inhibitors.

### Equivalents

Those skilled in the art will recognize, or be able to ascertain using no more than routine experimentation, many equivalents to the specific embodiments of the invention described herein. Such equivalents are intended to be encompassed by the following claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) and/or the National Center for Biotechnology Information (NCBI).

1. A composition comprising a Staphylococcus undecaprenyl pyrophosphate synthase in crystalline form, said synthase comprising an amino acid sequence at least about 80% homologous to SEQ ID NO:1.

2. The composition of claim 1 wherein said synthase comprises an amino acid sequence at least about 95% homologous to SEQ ID NO:1

3. The composition of claim 1 wherein the molecules of said synthase have atomic coordinates according to FIG. 5.

4. The composition of claim 1 wherein said synthase comprises at least one ligand.

5. The composition of claim 4 wherein the ligand is selected from the group consisting of farnesyl pyrophosphate, (S)-farnesyl thiopyrophosphate, isoprenyl pyrophosphate, magnesium ion, and sulfate ion.

6. The composition of claim 1 wherein said synthase comprises a first ligand binding site including the amino acid residues selected from the group consisting of Asp³³, Gly³⁴, Gly³⁶, Arg³⁷, Arg⁴⁶, Ala⁷⁶, Arg⁸⁴, Leu⁹⁵, Pro⁹⁶, and Phe¹⁴⁸.

7. The composition of claim 1 wherein the synthase comprises a first ligand binding site defined by amino acid residues Asp³³, Gly³⁴, Gly³⁶, Arg³⁷, Arg⁴⁶, Ala⁷⁶, Arg⁸⁴, Leu⁹⁵, Pro⁹⁶, and Phe¹⁴⁸ having atoms having atomic coordinates according to FIG. 5.

8. The composition of claim 1 wherein said synthase comprises a second ligand binding site including the amino acid residues selected from the group consisting of Asp³³, Arg²⁰¹, Arg²⁰⁷, Ser²⁰⁹, Glu²²⁰(B), and Gly²⁵¹(B).

9. The composition of claim 1 wherein the synthase comprises a second ligand binding site defined by amino acid residues Asp³³, Arg²⁰¹, Arg²⁰⁷, Ser²⁰⁹, Glu²²⁰(B), and Gly²⁵¹(B) having atoms having atomic coordinates according to FIG. 5.

10. A method of identifying a potential ligand for an undecaprenyl pyrophosphate synthase, comprising:
(a) using a three-dimensional structure of said synthase as defined by at least atomic coordinates of amino acid residues 33, 34, 36, 37, 46, 76, 84, 95, 96, and 148 according to FIG. 5;
(b) employing said three-dimensional structure to design or select said potential ligand;
(c) obtaining said potential ligand; and
(d) contacting said potential ligand with said synthase to determine binding of said potential ligand to said synthase.

11. The method of claim 10 wherein step (c) precedes step (b).

12. A method for identifying a potential inhibitor of a mutant undecaprenyl pyrophosphate synthase, said method comprising:
(a) using a three-dimensional structure of a known undecaprenyl pyrophosphate synthase as defined by atomic coordinates of said known synthase according to FIG. 5;
(b) replacing one or more amino acids of said known synthase, said amino acids selected from 33, 34, 36, 37, 46, 76, 84, 95, 96, 148, 201, 207, 209, 220, and 251 of SEQ ID NO:1, with a different naturally occurring amino acid, thereby forming said mutant synthase;
(c) employing said three-dimensional structure to design or select said potential inhibitor; and
(d) contacting said potential inhibitor with said mutant synthase, optionally in the presence of a substrate, to test the ability of said potential inhibitor to inhibit said mutant synthase.

13. A method of identifying a ligand capable of binding to an undecaprenyl pyrophosphate synthase substrate binding site, said method comprising:
(a) introducing into a suitable computer program information defining said binding site, said information comprising atomic coordinates of amino acids capable of binding to a synthase substrate, wherein said program displays the three-dimensional structure of said binding site;
(b) creating a three dimensional model of a test compound in the computer program;
(c) docking said model of said test compound to the structure of said binding site;
(d) comparing the docking of said model of said test compound to the docking of known ligands of said synthase to provide an output of the program.

14. A method for identifying a potential inhibitor for an undecaprenyl pyrophosphate synthase, said method comprising:
(a) using a three-dimensional structure of said synthase as defined by atomic coordinates of said synthase according to FIG. 5;
(b) employing said three-dimensional structure to design or select said potential inhibitor; and
(c) contacting said potential inhibitor with said synthase in the presence of a substrate to determine the ability of said potential inhibitor to inhibit said synthase.

15. A method of drug design comprising using atomic coordinates of a Staphylococcus undecaprenyl pyrophosphate synthase having at least one ligand binding site to computationally evaluate relative associations of chemical entities with the ligand binding site and produce an output.

16. A method for solving a crystal form comprising using atomic coordinates of a Staphylococcus undecaprenyl pyrophosphate synthase crystal or portions thereof, to solve a crystal form of a mutant, homolog or co-complex of the undecaprenyl pyrophosphate synthase by molecular replacement.

17. A machine-readable data storage medium comprising a data storage material encoded with machine-readable data comprising atomic coordinates comprising amino acid residues 33, 34, 36, 37, 46, 76, 84, 95, 96, and 148 according to FIG. 5.

18. The machine-readable data storage medium of claim 17 wherein said machine-readable data further comprises atomic coordinates comprising at least one amino acid residue selected from the group consisting of 201, 207, 209, 220(B), and 251(B) according to FIG. 5.

19. A computer-implemented tool for design of a drug, comprising:
(a) a three-dimensional structure of an undecaprenyl pyrophosphate synthase as defined by atomic coordinates of FIG. 5;
(b) a model of a chemical entity; and
(c) a computer program addressing said coordinates and capable of modeling the docking of said chemical entity in a ligand binding site of said three-dimensional structure to produce an output.

20. A computer for producing a three-dimensional representation of an undecaprenyl pyrophosphate synthase ligand binding site, said computer comprising:
(a) a machine-readable data storage medium comprising a data storage material encoded with machine-readable data comprising the atomic coordinates comprising the amino acid residues 33, 34, 36, 37, 46, 76, 84, 95, 96, and 148 according to FIG. 5;
(b) a working memory for storing instructions for processing said machine-readable data;
(c) a central-processing unit coupled to said working memory and to said machine-readable data storage medium for processing said machine readable data into said three-dimensional representation; and
(d) a display coupled to said central-processing unit for displaying said three-dimensional representation.
